(19) United States
(12) Patent Application Publication
(10) Pub. No.: US 2004/0030217 A1
(43) Pub. Date: Feb. 12, 2004

(54) Urethral muscle controlled micro-invasive sphincteric closure device

(76) Inventors: Jeffrey E Yeung, San Jose, CA (US); Teresa T Yeung, San Jose, CA (US)

Correspondence Address: Jeffrey E Yeung, 834 North White Road, San Jose, CA, 95127, US

(21) Appl. No.: 10/399,707

(22) PCT Filed: Feb. 2, 2001
(86) PCT No.: PCT/US01/03513

(51) Int. Cl. A61F 2/02

(52) U.S. Cl. 600/31; 606/185

(57) ABSTRACT

A rod-like or tube-like sphincteric closure device is micro-invasively implanted beneath the surface of the posterior mucosal wall to elevate the mucosa and close the lumen, by using a delivery device operated through the urethra. To initiate voiding, the muscle-rich anterior urethral wall contracts and widens the lumen beyond the closing or elevating range of the sphincteric closure device. For urethral obstruction, repelling magnets are micro-invasively implanted beneath the surface of mucosa with the delivery device through the urethra, to widen the lumen with magnetic force.

### FIELD OF THE INVENTION

This invention relates to rod-like or tube-like sphincteric closure devices implanted beneath the surface of the mucosa through the urethra with a micro-invasive delivery device. The sphincteric closure devices narrow or close the lumen opening to prevent urine leakage without interfering with the process of voluntary voiding.

### BACKGROUND, TRADITIONAL TREATMENTS AND PRIOR INVENTIONS

### Prevalence and Cost of Urinary Incontinence

Urinary incontinence is one of the most common urinary dysfunctions. The number of people living with urinary incontinence is far higher than estimated, even by most primary care physicians. A report published by the Agency for Health Care Policy and Research of the U.S. Public Health Service estimates that at least 10 million, more likely 20 million, adult Americans are affected by urinary incontinence. Many patients, especially women, do not mention their incontinence problems to their physicians. One of the reasons is that women are accustomed to using feminine hygiene products, some of which are designed for urine absorption. Among the elderly population, a 1975 report from the U.S. Department of Health showed that 55% of the surveyed patients living in long-term facilities had problems with urinary control. In 1980, a large European postal survey of 22,430 people from ages 5 to over 85 showed that up to 8.5% of surveyed individuals had two or more episodes of urinary incontinent occurrences in a month. The percentage of women within the age groups who suffer from occasional incontinence is much higher (Raz S., Female Urology, 2nd Ed., W. B. Saunders Co., 1996, pp.73-74).

Although urinary incontinence is not a life threatening disease, many incontinent patients suffer intense embarrassment, loss of self-esteem, feelings of helplessness, limitations on travel, depression, anxiety, avoidance of sexual relationships and withdrawal from social contacts (Urology Times, February 1996; Hu T-W., J. Am. Geriatr. Soc., 38:292, 1990).

Urinary incontinence is costly to patients and health care systems. Annual sales of adult disposable diapers reach half a billion dollars. The annual cost to the U.S. health care system for treating patients suffering from urinary incontinence exceeds $15 billion, according to a 1996 report “Urinary Incontinence in Adults Acute and Chronic Management”, published by the Department of Health and Human Services. The indirect cost is likely to be much higher, in fact incalculable. The careers of the sufferers are often prematurely terminated or adversely affected by the offensive odor. The financial and social impact from urinary incontinence are very real, significant and rapidly growing as our population ages.

### Mechanism of Urethral Sphincter

The two major urinary closures in our bodies are the urethral sphincter and the bladder neck. The urethral sphincter is often perceived as a mechanical valve, which stops the flow of urine from the bladder. However, unlike the valves of a heart, the urethral sphincter cannot be identified with the naked eye or even under a microscope. The interior layer of the urethra is an integrated interaction between smooth and striated muscle with collagen and elastin forming spongy and supple mucosal folds, which actuate the closure of the urethral lumen. The exterior or outer layer of the urethra provides structural and ligamental support. The external striated layer of the urethral sphincter consists of bundles of circularly arranged fibers with maximal density at the mid-urethral level anteriorly, thinning laterally and becoming almost totally deficient posteriorly (Gosling J. A., et al., J. Anat. 129:216, 1979; Stanton S. L., et al., Surgery of Female Incontinence, 2nd Ed., Springer-Verlag, NY., 1986, pp. 4-5). Slow-twitch muscle fibers primarily provide involuntary urinary sphincteric control; fast-twitch fibers are responsible for voluntary sphincteric activity. Therefore, the sphincter is under partial voluntary control. (Raz S., Female Urology, 2nd Ed., W. B. Saunders Co., 1996, pp. 58-59).

The female urethra is between 30 and 50 nmm in total length, including the sphincteric length of about 28 mm, with the lumen diameter being about 5.3 mm. The sponge-like folding and suppleness of the resilient mucosa are promoted and maintained by sex hormone. With age and the declining level of sex hormone, the mucosa of the middle and proximal portions of the urethra thins out (Stanton S. L., et al., Surgery of Female Incontinence, 2nd Ed., Springer-Verlag, NY, 1986, p. 5).

During stress from suddenly increasing abdominal pressure, such as coughing or sneezing, the tensile forces of the urethropelvic ligaments pull on the urethra laterally and collapse the opening of the lumen, as indicated in FIG. 2. The spongy mucosa in the lumen forms a coaptive seal to prevent urine leakage (Raz S., Female Urology, 2nd Ed., W. B. Saunders Co., 1996, p. 66).

On the other hand, voiding is accomplished by the relaxation of pelvic floor muscles, contraction of detrusor muscles from the abdomen and increased tension of urethral muscles to shorten and widen the urethra (Lapides J., J. Urol. 80:341-353, 1958; Bradley W. E., et al., Urol. Clin. North. Am., 1: 3-27, 1974; Stanton S. L., et al., Surgery of Female Incontinence, 2nd Ed., Springer-Verlag, NY, 1986, p. 11).

Urinary control is a complex mechanism that involves the bladder neck, proximal urethral smooth muscle and anatomic support of both the bladder base and urethra. Circular fibers of smooth muscle are found in the bladder neck. It seems that passive elastic tension is the most important factor leading to closure of the bladder neck and proximal urethra.

The bladder neck and the proximal urethra retain sphincteric function unless they are damaged by disease, surgery, pregnancy or by the constant pull of gravity on the muscular and ligamental supports (Campbell's Urology, Ed. P. Walsh et al., 7th Ed., Vol. 1, 1998, p.1007).

### Factors Leading to Urinary Incontinence

The following elements are essential for apposition and coaptation of the mucosa: urethral wall tension, external compression, urethral support, adjustment during increased abdominal pressure and suppleness of the mucosa (Zinner N. R., et al., Urology, 1980, 16:115; Campbell's Urology, Ed. P. Walsh et al., 7th Ed., Vol. 1, 1998, p.1017).

It is widely believed that a leading cause of urinary incontinence is the loss of structural support for the urethra, especially behind the posterior urethral wall, which is indicated by hypermobility of the urethra. Gravity and/or pregnancy may adversely affect the structural support. As a result, descent of the bladder neck and urethra in varying degrees lead to varying types of stress incontinence (Campbell's Urology, Ed. P. Walsh et al., 7th Ed., Vol. 1, 1998, p.1018; Walters M. D., J. Repro. Med. 1990, 35(8): 777-784).

The structural (anatomic) support of the muscle-poor posterior urethral wall serves as a backboard against which the urethra is compressed during increased abdominal pressure. Research studies using magnetic resonance imaging substantiate the importance of posterior support of the urethra. During stress in the incontinent patient, there is an unequal movement of the anterior and posterior walls of the vesical (bladder) neck and urethra proximal to the bladder. The urethral lumen is actually pulled open as the posterior wall moves away from the anterior wall; then leakage occurs (Mostwin J. L. et al., Urol. Clin. North. Am. 1995, 22(3): 539-549; Campbell's Urology, Ed. P. Walsh et al., 7th Ed., Vol. 1, 1998, p. 1018).

In men, sphincteric abnormalities are most commonly caused by anatomic disruption after prostate surgery, trauma or neurologic abnormalities. After radical prostatectomy, five to ten percent of the patients suffer from permanent urinary incontinence. In women, sphincteric abnormalities may be classified in two ways: (1) urethral hypermobility, and (2) intrinsic sphincter deficiency. Urethral hypermobility is often caused by a weakness of pelvic floor support. During an increase in abdominal pressure, vesical neck and proximal urethra rotationally descend and slip away from the posterior support. (Campbell's Urology, Ed. P. Walsh et al., 7th Ed., Vol. 1, 1998, pp.1011-1012).

Incontinence that occurs during stress is not always caused by the lack of anatomic support or sphincteric abnormalities. In some patients, stress initiates an abdominal detrusor contraction. This condition has been called stress hyper-reflexia. Stress incontinence and hyper-reflexia are easily differentiated. If the leakage stops as soon as the stress is over, it is stress incontinence. If voiding uncontrollably follows the stress, it is hyper-reflexia or detrusor hyperactivity, a common condition especially among the elderly (Campbell's Urology, Ed. P. Walsh et al., 7th Ed., Vol. 1, 1998, p.1023; Raz p. 231).

In one study of incontinent women, 38% had mixed hyper-reflexia and stress incontinence, and 16.5% had hyper-reflexia alone as the cause of the incontinence (Sand P. K., Obstet. Gynec., 70:57, 1987). Although genuine stress incontinence is probably the most common cause of urinary incontinence in women, the incidence and prevalence of detrusor hyperactivity increases with age (Bates C. P., Clin. Obstet. Gynecol., 5:109, 1978).

### Diagnosis of Urinary Incontinence.

Physical examination, urodynamics (study of urine propulsion and flow) and cystoscopy (endoscopy for the urinary tract) are commonly used to determine the true nature of the patient's stress incontinence and to guide in the choice of treatment.

To determine urethral hypermobility, a cotton-swab test is used in physical examination. A well-lubricated and sterile cotton-swab is inserted into the urethra. During coughing, an unstable urethra sways and is evident by the outer portion of the cotton swab. If the sway is greater than 15 degrees, the patient has urethral hypermobility.

Cystometry is a urodynamic method used to measure intravesical bladder pressure during the course of bladder filling. The filling medium may be carbon dioxide or a liquid, such as water, saline or radiographic contrast material. Pressure is measured during and after filling (Campbell's Urology, Ed. P. Walsh et al., 7th Ed., Vol. 1, 1998, p.934). With about 200 ml filing medium in the bladder and about 55-cm water pressure, stress is initiated. If voiding stops at the end of the stress, the patient has stress incontinence, which indicates intrinsic sphincter deficiencies (ISD). If voiding continues after the stress ceases, it is likely detrusor hyperactivity, or hyper-reflexia. To determine the degree of incontinence, the fill volume and pressure is increased to the point where involuntary voiding occurs; this is defined as the leak-point pressure in urodynamics.

### Classification of Stress Incontinence

To evaluate the degree of bladder/urethral support and sphincter competence, stress incontinence is divided into the following five classifications. Type 0: Patient complains of stress urinary incontinence. Videourodynamic testing reveals that both the vesical neck and proximal urethra are closed at rest and situated at or above the lower end of the pubis symphysis. During stress, the vesical neck and proximal urethra descend and open, assuming an anatomic configuration similar to that seen in types I and II stress urinary incontinence, but with no urine leakage. Type I: The vesical neck is also closed at rest and situated above the inferior margin of the pubis symphysis. During stress, the vesical neck and proximal urethra open and descend less than 2 cm. Urinary incontinence is apparent with increased abdominal pressure. Type IIA: The vesical neck is also closed at rest and situated above the inferior margin of the pubis symphysis. During stress, the vesical neck and proximal urethra are also open, but with a rotational descent characteristic of a cystourethrocele (prolapse of bladder and urethra) which accompanies urine leakage. Type IIB: The vesical neck is closed at rest but situated at or below the inferior margin of the pubis symphysis. During stress, there may or may not be further descent, but the proximal urethra opens and incontinence ensues. Type III: The bladder neck and urethra are open at rest indicating intrinsic sphincter dysfunction with or without hypermobility. Obvious urinary leakage is associated with minimal abdominal pressure (Campbell's Urology, Ed. P. Walsh et al., 7th Ed., Vol. 1, 1998, pp.1013-1016; Raz S., Female Urology, 2nd Ed., W. B. Saunders Co., 1996, p.345).

### Non-Surgical Treatments

Non-surgical treatments include (1) pelvic floor exercise to strengthen pelvic muscles, (2) estrogen to thicken mucosa, (3) biofeedback and/or electrical stimulation to stimulate certain sets of urethral muscles, (4) alpha-sympathomimetic drugs for intrinsic sphincter deficiency, and (5) mechanical devices to clamp the urethra.

Pelvic floor exercise and estrogen may have value as preventive measures. Biofeedback and electrical stimulation have been reported to cause improvement in 30% to 75% of patients; but only about 10% of patients experience a “cure” with little long-term data confirming the claims. Drug therapy has very limited success and significant side effects.

Urethral removable plugs (U.S. Pat. No. 5,562,599 to Beyschlag, U.S. Pat. No. 4,457,299 to Cornwell, U.S. Pat. No. 5,131,906 to Chen, U.S. Pat. No. 5,906,575 to Conway et al., U.S. Pat. No. 5,885,204 to Vergano) are uncomfortable and troublesome to use, and their use increases the possibility of urinary tract infections. Penile clamping devices (U.S. Pat. No. 4,942,886 to Timmons) are also highly uncomfortable and unnatural and may even cut off blood supply. For females, pessary devices (U.S. Pat. No. 5,007,894 to Enhorning, U.S. Pat. No. 5,386,836 to Biswas, U.S. Pat. No. 5,785,640 to Kresch et al.) are designed to be worn in the vagina to compress and stop the leakage of urine. To be effective, the compression has to be strong and uncomfortable. Similar to the urethral plugs, pessary devices increase the possibility of infections and are troublesome to use, messy during menstrual periods.

### Surgical Treatment

In general, surgical treatments for urinary incontinence are far more successful than existing non-surgical treatments and are the only reasonable long-term solution thus far. The primary goals of the surgical approaches for sphincteric incontinence are (1) to correct urethral hypermobility and the excessive anatomic descent of the bladder neck/urethra, and (2) to increase urethral resistance by improving urethral coaptation and compression for treating intrinsic sphincteric dysfunction (Campbell's Urology, Ed. P. Walsh et al., 7th Ed., Vol. 1, 1998, p. 1018, p.1066). Surgical procedures designed to meet these two simple goals differ in their suture material, placement, depth, distance from urethra and location of abdominal anchoring sites.

For anatomic corrections, sutures are used to pull and lift the vaginal wall forward and upward along with the urethra and bladder neck. In essence, the vaginal tissue serves as the supporting backboard for the urethra. Sutures are then fastened onto abdominal tissue or the pubis symphysis. The major differences between surgical procedures of this type are the location of incisions, vaginal suspension, transvaginal suspension, and requirement of tissue dissection.

Burch and Marshal-Marchetti-Krantz procedures use the vaginal-abdominal approach requiring abdominal incisions; while Raz suspensions, Stamey needle and Gittes needle are the transvaginal suspension procedures. Some surgeons prefer opening both abdominal and vaginal cavities.

Several less invasive needles and devices (U.S. Pat. No. 5,860,425, U.S. Pat. No. 5,836,314 to Benderev et al., U.S. Pat. No. 5,816,258 to Jervis, U.S. Pat. No. 5,697,931 to Thompson, U.S. Pat. No. 5,647,836 to Blake and U.S. Pat. No. 5,549,617 to Green et al.) are designed to pull the urethra forward by pulling the vaginal anterior wall forward. Without a direct view of the surgical site, one of the major potential problems with the devices is the uncertainty of suture tension, let alone obtaining the optimal suture tension. If the suture is too tight, the urethra is too restricted, and urinary obstruction occurs. Removing existing sutures with surrounding fibrotic tissue formation is an invasive surgery. If the tension is too loose, incontinence continues.

Common anatomic surgical complications include recurrent or persistent urinary incontinence, irritation, urinary retention, obstruction and/or persistent postoperative pain, which may be caused by urethral kinking, improper suture placement or improper tension. Other complications, such as wound infection, abscess formation, genitofemoral nerve entrapment, bladder leakage or urethral damages, are common occurrences as well (Campbell's Urology, Ed. P. Walsh et al., 7th Ed., Vol. 1, 1998, p.1100). The overall complication rate ranges from 3% to 32% (Campbell's Urology, Ed. P. Walsh et al., 7th Ed., Vol. 1, 1998, p.1101). Furthermore, due to depth and axis alteration, numerous vaginal posterior prolapses have been reported following anatomic correction (Langer R. et al., Obstet. Gynecol. 1988, 72:866-869; Wiskind A. K., et al., Am. J. Obstet. Gynecol., 1992: 167:399-405; Campbell's Urology, Ed. P. Walsh et al., 7th Ed., Vol. 1, 1998, p.1101).

For intrinsic sphincter dysfunction, merely anatomic correction supported by a wall of soft vaginal tissue is inadequate. Therefore, sling procedures have been designed to loop behind the urethra and fasten onto the abdominal tissue. The loop forms a relatively firm backboard, which compresses and restricts the urethral sphincter. Slings are also effective in treating neurogenic intrinsic sphincter deficiency, such as myelodysplasia, a defective development of the lower segment of the spinal cord, (Gormley E. A., J. Urol. 1994, 152:822; McGuire E. J., J. Urol., 1987, 138:525-526; McGuire E. J., J. Urol., 1986, 135:94). A less invasive sling needle (U.S. Pat. No. 5,899,909 to Claren et al.) has been invented to treat female sphincteric deficiency. Another invention (U.S. Pat. No. 5,934,283 to Willem et al.) utilizes various materials, including autologous, heterologous or artificial material to construct a sling.

Common complications of the slings include sensations of inguinal pulling, potential erosion of the urethra, urinary retention, urethral obstruction and enterocele (posterior vaginal hernia). Most of these complications are once again due to improper tension of the suture or sling. If the sling is too tight, the urethra is obstructed; if it is too loose, incontinence continues. Unfortunately, no standard parameters exist to identify the appropriate sling tension. Thus, it remains more an art than a science, with a limited margin of error.

In many failed sling procedures in the past, sutures attaching the urethra to the abdominal ligaments were too close to the urethra. Due to the close proximity of the suture and the pliable nature of the urethra, the tension of the suture created kinks in the urethra, resulting in urinary obstruction. Furthermore, the rubbing of the abdominally anchored suture onto the urethra is presumably the cause of fibrotic tissue formation around the urethra and sometimes urethral erosion to the point of severance.

Two other techniques, injectable materials and artificial sphincters, are often used to treat intrinsic sphincter deficiency. Injectable or bulking agents, such as collagen, polytetrafluoroethylene (PTFE), autologous fat and silicone, are injected into the wall of the bladder neck or urethral mucosa to decrease the size of the lumen opening and provide a more manageable or controllable sphincter. However, multiple injections are usually necessary for achieving noticeable improvement, especially in males. Furthermore, all these bulking agents migrate or metabolize away, some in less than a few months. Collagen begins degradation in twelve weeks. PTFE migrates and granuloma forms (Malizia A. A. Jr., et al., JAMA 1984, 251:3277-3281). Silicone polymers migrate and deposit in the lungs, kidneys, brain and lymph nodes.

Usually when all else fails in treating intrinsic sphincter deficiency, an artificial sphincter is implanted beneath the bladder neck around the urethra to mechanically pinch or restrict the opening of the lumen. Numerous artificial sphincters (U.S. Pat. No. 5,893,826 to Salama, U.S. Pat. No. 5,704,893 to Timm, U.S. Pat. No. 5,562,598 to Whalen et al., U.S. Pat. No. 5,097,848 to Schwarz, U.S. Pat. No. 4,994,020 to Polyak, U.S. Pat. No. 4,705,518 to Baker et al., U.S. Pat. No. 4,632,114 to Todd et al. and U.S. Pat. No. 4,552,128 to Haber) have been designed to restrict the urethra mechanically.

Implantation of an artificial sphincter is invasive surgery. Typically, an inflatable cuff is inserted around the bulbous urethra in the male or the bladder neck in the female. The tubing, liquid reservoir and pumps are implanted in the abdomen. Hospital post-surgical care lasts around three days.

Post-surgical complications include hematoma, cuff erosion, tissue atrophy, early infection from surgical contamination, late infection from urinary tract origin and mechanical malfunction, such as tube kinking or leaks (Carson C. C., Urol. Clin. North. Am, 1989, 16:139-147). Tissue atrophy, a natural result of cuff compression over time, is often followed by cuff erosion with symptoms of pain, swelling, infection and/or bloody discharge. Confirmation of erosion mandates cuff removal (Campbell's Urology, Ed. P. Walsh et al., 7th Ed., Vol. 1, 1998, p.1131-1132).

To maximize the longevity of the artificial sphincter, multiple life-long restrictions, which includes deactivation of the sphincter as often as possible and avoidance of bicycle riding, horseback riding and prolonged sitting are imposed. Furthermore, during pregnancies, the sphincter needs to be deactivated during the last trimester, and delivery by cesarean section is strongly recommended (Barrett D. M., et al., Urol. Clin. North. Am., 1989, 16:119-132; Campbell's Urology, Ed. P. Walsh et al., 7th Ed., Vol. 1, 1998, p.1111 and p.1130-1131).

### Urethral Obstruction

One of the most common urinary dysfunctions among middle aged and elderly men is urethral obstruction; and the most common cause of the obstruction is lumen narrowing of the supple urethra by an enlarged prostate, a condition called benign prostatic hyperplasia (BPH). Two classes of drugs are available to ease the urethral blockage. Alpha-blockers, such as phenoxybenzamine, prazosin, terazosin and doxazosin, are used to relax smooth muscles such as the one around the prostate, thus minimizing the restriction around the urethra. However, alpha-blockers have the side effect of hypotension, characterized by dizziness. Within the androgen suppression class of drugs, Finasteride is the only one with clinically acceptable tolerance. Androgen suppression causes a reduction in prostate volume, hence reducing the obstruction around the urethra. The primary side effects of androgen suppression are impotence and decreased ejaculatory volume.

Urethral stents, the only non-invasive device, are used to open the restricted urethra within the benign prostatic hyperplasia. However, given time, epithelial tissues grow into the lumen of the stents, requiring traumatic surgical removal. Several minimally invasive treatments, including high intensity ultrasound, laser, hyperthermia, thermotherapy, electro-vaporization, radio-frequency ablation and balloon dilation, have been invented for BPH. However, surgical transurethral resection of the prostate has been and still is the gold standard in terms of improving flow rate and decreasing postvoid residual urine.

### SUMMARY OF INVENTIONS

A rod-like and a tube-like sphincteric closure devices are implanted beneath the surface of he urethral mucosa with a delivery device using micro-invasive methods to elevate the mucosa and close the lumen of the urethra, without interfering with the voluntary muscular contraction for voiding. To urinate, the muscle-rich anterior urethral wall stiffens and widens to enlarge the lumen beyond the range of elevation from the sphincteric closure devices.

One of the sphincteric closure devices is made with two attracting magnets sandwiching the lumen to assist with lumen closure. By reversing the polarity of one of the magnetic devices, the magnetic devices repel each other from beneath the mucosa to open the lumen and push against urethral obstruction, such as that found in benign prostatic hyperplasia.

### Arch Closure Device

An arch is made with elastic or shape memory material capable of being resiliently straightened in a needle equipped with a plunger for delivery. To assure that the curvature of the arch is bent toward the proper direction during delivery, the arch and the passage of the needle can be made non-round or elliptical to prevent arch rotation within the needle.

The arch, needle and plunger assemblies are parts of a delivery device with a balloon behind an indented pocket or recessed region. After insertion of the device into the urethra, the balloon is inflated from behind the indented recess, pushing and embedding the recess into the mucosal tissue. The indented recess holds or shelters a portion of mucosa and other soft urethral tissue from being flattened by the compression. The needle and the arch are then advanced through a hole in the proximal recessed wall to longitudinally penetrate beneath the surface of the loosely packed mucosa within the recessed pocket. To deploy the resiliently straightened arch, the needle is withdrawn while the plunger is held stationary behind the arch. As a result, the arch resumes the pre-disposed curvature toward the lumen, beneath the surface of the mucosa. After the balloon is deflated, the delivery device is withdrawn. The curvature or bend of the arch protrudes from within the urethral wall, elevating or lifting the mucosa to narrow or close the lumen, preventing or minimizing urine leakage. For voiding, the urethral muscles stiffen the urethra and significantly widen the lumen beyond the closing range of the arch. As a result, urine passes.

### Arch Tube Closure Device

An arch tube is also made with elastic or shape memory material capable of being resiliently straightened by a rigid trocar passing through its passage for delivery. To ensure the curvature of the arch tube is bending toward the proper direction during delivery, the trocar and the passage of the arch tube can also be made non-round or elliptical to prevent rotation of the arch tube around the trocar.

The trocar and the arch tube are loaded as parts of a delivery device, similar to the one previously mentioned, equipped with a balloon behind an indented pocket or recessed region. The balloon is used to compress or position the recess into the soft urethral tissue, holding and sheltering the mucosa from flattening. The trocar is then advanced from a hole in the recess wall, penetrating beneath the loosely packed urethral tissue in the recess. The resiliently straightened arch tube is slid into position over the deployed trocar, both beneath the surface of the mucosa. While holding the arch tube stationary, the trocar is withdrawn, allowing the arch tube to resume the pre-disposed curvature toward the lumen beneath the mucosa. After the balloon is deflated, the delivery device is withdrawn. Similar to the arch, the arch tube also curves or bends toward the lumen, lifting the mucosa upward to coapt, narrow or close the lumen of the urethra, thus preventing or minimizing urine leakage.

### Swellable Closure Device

A tube-like or rod-like swellable device is implanted beneath the surface of the mucosa with the delivery device. After hydration within the urethral tissue, the swollen device increases greatly in size, pressing against the surrounding tissue, especially into the vacant lumen space. As a result, the lumen is significantly narrowed or closed by the swollen device.

### Inflatable Closure Device

Similar to the swellable device, an inflatable closure device is implanted beneath the surface of the mucosa with the delivery device. After the device is inflated by air, gas or liquid, it expands and pushes upon the surrounding tissue, particularly toward the direction of the vacant lumen space, to narrow or close the lumen.

### Magnetic Closure Device

Two magnets with attractive polarities are implanted with the delivery device beneath the mucosa, one on each side of the urethra, sandwiching and compressing the lumen to improve closure and minimize urine leakage. Due to the relatively small urethral diameter, the magnetic forces created by the implanted magnets are strong and can be effectively assisting lumen closure.

### Retrieval of Sphincteric Closure Device

For some adverse events, retrieval of the sphincteric closure device may be necessary. For ease of retrieval, a suture can be attached to the proximal end of the device. By pulling on the suture, the closure device can slip out from the soft urethral tissue, without invasive surgery. The biodegradable suture will be eliminated by cutting, urine excretion and/or biodegradation, while the sphincteric closure device remains in place.

### Urinary Obstruction

Most of the urinary obstruction in men is caused by the enlargement of the prostate encroaching and pinching both the urethra and lumen opening. By reversing the polarities of the magnets, instead of attraction for lumen closure, the magnets repel each other, pushing the urethral wall outward to open the lumen, restoring urine flow.

| | REFERENCE NUMBERS |
|---|---|
| Suture | 21 |
| Lumen | 100 |
| Urethra | 101 |
| Urethropelvic ligament | 102 |
| Submucosa | 103 |
| Arch tube | 104 |
| Smooth muscle | 105 |
| Elastic curvature | 106 |
| Delivery device | 107 |
| Striated muscle | 108 |
| Trocar | 109 |
| Trocar advancer | 110 |
| Bladder | 111 |
| Bladder neck | 112 |
| Mucosa | 113 |
| Vagina | 114 |
| Pubis symphysis | 115 |
| Rectum | 116 |
| Urine | 117 |
| Anterior urethral wall | 118 |
| Posterior urethral wall | 119 |
| Device advancer | 120 |
| Suture knot | 121 |
| Indented panel | 122 |
| Recess or Pocket | 123 |
| Prostate | 124 |
| Deployment opening | 125 |
| Receiving opening | 126 |
| Balloon | 127 |
| Tubing | 128 |
| Delivery device penetration marker | 129 |
| Delivery device orientation line | 130 |
| Lateral urethral wall | 131 |
| Magnet | 132 |
| Coating | 133 |
| Swellable closure device | 134 |
| Inflatable closure device | 135 |
| Stem | 136 |
| Detachable tube | 137 |
| Endoscope | 138 |
| Needle | 139 |
| Bulking agent | 140 |
| One-way valve | 141 |
| Recess positioner | 142 |
| Recess positioner handle | 143 |
| Passage of arch | 144 |
| Sling pad | 145 |
| Urethral support | 146 |
| Detrusor contraction | 147 |
| Pivotal leg of arch | 148 |
| Anchoring device | 149 |
| Tissue ingrowth opening | 150 |
| Arch | 151 |
| Needle | 152 |
| Plunger | 153 |
| Needle advancer | 154 |
| Plunger holder | 155 |
| Anterior side of the arch | 156 |
| Posterior side of the arch | 157 |
| Lateral side of the arch | 158 |
| Panel-restricting tube | 159 |
| Receiving trough | 160 |

### BRIEF DESCRIPTION OF DRAWINGS

FIG. 1 indicates a normal, well-supported bladder 111 in dashed lines and a descended bladder 111 with a widened bladder neck 112 in solid lines.

FIG. 2 shows a failed lumen 100 closure and hypermobility under stress with the urethropelvic ligament 102 pulling the lateral walls 131 of the poorly supported urethra 101.

FIG. 3 indicates a mid-longitudinal view of FIG. 2 and urine 117 leakage during stress with urethropelvic ligaments pulling perpendicularly above and below the plane of the page.

FIG. 4 shows a traditional surgical procedure for the treatment of urinary incontinence, using sutures 21 to pull the vagina 114 forward, supporting or gently compressing the urethral posterior wall.

FIG. 5 depicts a section of the surgically corrected urethra 101 with sutures 21 pulling the vaginal 114 tissue to support and gently compress the urethral posterior wall 119.

FIG. 6 indicates the lumen 100 closure of the surgically corrected urethra 101 under stress, with urethropelvic ligaments 102 pulling the lateral walls 131 of the supported urethra 101.

FIG. 7 shows another surgical approach to narrowing the enlarged opening of the bladder neck 112 by looping a padded 145 suture 21 sling around the neck 112.

FIG. 8 depicts the injection of bulking agent 140 within the wall of the urethra 101 and bladder neck 121 to narrow the lumen 100, monitored by an endoscope 138.

FIG. 9 indicates an arch 151 composed of legs 148 and an elastic or shape memory curvature 106, with length L, height of curvature, H, and diameter, D.

FIG. 10 shows the resiliently straightened arch 151 in a needle 152 with a plunger 153.

FIG. 11 depicts the deployment of the arch 151 by withdrawing the needle 152 while holding the plunger 153 stationary behind the arch 151. The arch 151 resumes the pre-disposed curvature 106.

FIG. 12 indicates a delivery device 107 equipped with an arch 151 and a plunger 153 in a needle 152 adjacent to a tubing 128 for inflating and deflating a balloon 127.

FIG. 13 shows the inflated balloon 127 behind an indented recess 123 of the delivery device 107.

FIG. 14 depicts the advancement of the needle 152 with the arch 151 through the recess 123, by pushing a needle advancer 154.

FIG. 15 indicates the deployment of the arch 151 by withdrawing the needle 152 while the plunger 153 is held stationary behind the arch 151. The arch 151 then resumes the pre-disposed curvature 106.

FIG. 16 shows the deflation of the balloon 127.

FIG. 17 depicts a cross-section of a urethra 101 with multiple layers of tissue.

FIG. 18A shows the insertion of the delivery device 107 into a urethra 101 before inflating the balloon 127. A cross-section is marked 18B, as shown in FIG. 18B.

FIG. 18B depicts the cross section of a urethra 101 showing the mid-cross-section of the indented panel 122 and the balloon 127, as marked in FIG. 18A.

FIG. 19A indicates the inflated balloon 127 compressing the ends of the indented recess 123 into the urethral wall. Within the recess 123, the mucosa 113 is sheltered from compression, shown as a plane of mucosa 113 in a rippled dotted line around the recess 123.

FIG. 19B shows a cross section of FIG. 19A with the inflated balloon 127 pushing the recess into submucosa 103 to store and loosely pack the soft urethral 101 tissue on the indented panel 122.

FIG. 20A indicates the penetration of the needle 152 with the arch 151 beneath the surface of loosely packed mucosa 113 in the recess 123.

FIG. 20B depicts the cross section of FIG. 20A with the arch 151 inside the needle 152 advanced into the smooth muscle 105 within the recessed region.

FIG. 21A shows the withdrawal of the needle 152 by retracting the needle advancer 154 while the plunger 153 is held stationary, allowing the curvature 106 to elevate the mucosa 113 toward the panel 122.

FIG. 21B indicates a cross section of FIG. 21A with the arch 151 lifting the submucosa 103 and smooth muscle 105 toward the panel 122 of the recessed region.

FIG. 22A indicates a deflated balloon 127 and the deployed arch 151 implanted beneath the surface of mucosa 113, detached from the delivery device 107.

FIG. 22B shows a cross section of FIG. 22A, indicating the lifting of the smooth muscle 105 and submucosa 103 by the arch 151 toward the center of the urethra 101.

FIG. 23A depicts the arch 151 implanted beneath the surface of mucosa 113 bending to close the lumen 100 after withdrawal of the delivery device.

FIG. 23B indicates a cross section of FIG. 23A showing lumen 100 closure by the lifting of the arch 151 upon the mucosa 113, submucosa 103 and smooth muscle 105.

FIG. 24 depicts the activation of detrusor contraction 147 and urethral 101 muscles to widen the lumen 100 beyond the elevated range of the arch 151 to void.

FIG. 25 shows a delivery device 107 with orientation line 130 and penetration markers 129.

FIG. 26 depicts a delivery device 107 with two deployment openings 125 for passage of two needles 152 housing arches 151 within them.

FIG. 27 indicates a cross section of a closed lumen 100 with a large portion of urethral 101 tissue lifted by two arches 151 to ensure lumen 100 closure.

FIG. 28 depicts an arch 151 with a suture 21 attached.

FIG. 29 shows anchoring devices 149 on an arch 151 for maintaining position and preventing migration.

FIG. 30A indicates an arch 151 designed with lateral edges 158 for tissue anchoring to resist rotation of the curvature 106 away from the lumen. A cross section is shown in FIG. 30B.

FIG. 30B depicts a cross section of FIG. 30A with lateral edges 158 for tissue anchoring and a mucosal lifting anterior 156 side.

FIG. 31 shows a cross section of a curvature of another arch 151 with a tissue-trapping anterior 156 valley to prevent shifting of the curvature from facing the lumen to rotating to the side.

FIG. 32 indicates an arch 151 with tissue ingrowth openings 150 to resist curvature 106 shifting or arch 151 migration with time.

FIG. 33 depicts an arch 151 with a narrow curvature 106 and wide legs 148 to further stabilize the implant.

FIG. 34A shows a resiliently straightened or flattened curvature 106 within the body of an arch 151.

FIG. 34B indicates a deployed curvature 106 protruding from the body of the arch 151.

FIG. 35 indicates an arch tube 104 with legs 148, an elastic or shape memory curvature 106 and a central passage 144. The dimensions are length, L, height of curvature, H, and diameter, D.

FIG. 36 shows the resilient arch tube 104 straightened by the insertion of a rigid trocar 109.

FIG. 37 depicts a delivery device 107 equipped with the arch tube 104, trocar 109 and tubing 128 for inflating and deflating a balloon 127.

FIG. 38 indicates the inflated balloon 127 behind an indented recess 123 of the delivery device 107.

FIG. 39 shows the deployment of the trocar 109 through the indented recess 123 by pushing in the trocar advancer 110.

FIG. 40 depicts the advancement of the resiliently straightened arch tube 104, pushed by a device advancer 120, sliding over the trocar 109 into the recess 123.

FIG. 41 shows the withdrawal of the trocar 109 while holding the device advancer 120 stationary to release the arch tube 104, allowing the arch tube 104 to resume the pre-disposed curvature.

FIG. 42 indicates a suture 21 attached to an arch tube 104.

FIG. 43 shows an elliptical arch tube 104 used to elevate mucosa to close the lumen.

FIG. 44 indicates an arch tube 104 with a lengthened elastic curvature 106 to increase the length of sphincteric closure action.

FIG. 45 depicts double elastic curvatures 106 to double the sphincteric closure action.

FIG. 46 shows triple closure action from both legs 148 and the curvature 106.

FIG. 47 indicates an elastically curved 106 strip for elevating mucosal tissue to close the lumen.

FIG. 48 shows a modular arch tube 104 to accommodate length-wise movement of the urethra.

FIG. 49 depicts a urethral support 146 to strengthen the urethral wall.

FIG. 50 shows a swellable closure device 134 around a trocar 109.

FIG. 51 indicates a cross section of a urethra 101 implanted with a swellable closure device 134 beneath the mucosal 113 surface.

FIG. 52 depicts the enlargement of the swollen closure device 134 after absorption of water or blood serum.

FIG. 53 shows a cross section of lumen 100 closure from compression by the enlargement of the swollen closure device 134.

FIG. 54 indicates an inflatable implant 135 around the trocar 109.

FIG. 55 depicts the inflated implant 135 with a one-way valve 141 within a stem 136 connected to a detachable tube 137.

FIG. 56 indicates a mid-longitudinal view of a recess positioner 142 above the delivery device 107, positioned for urethral insertion.

FIG. 57 shows the position of the recess positioner 142 for pushing the recess 123 of the delivery device 107 into the mucosal tissue.

FIG. 58 shows a resilient panel 122 outside a panel-restricting tube 159.

FIG. 59 indicates the resiliently straightened panel 122, shown in FIG. 58, within the panel-restricting tube 159.

FIG. 60 depicts the delivery of the resiliently straightened panel 122 in the panel-restricting tube 159 into the urethra 101.

FIG. 61 shows the deployment of the resilient panel 122 with distal and proximal ends of the recess 123 embedded beneath the surface of mucosal 113 tissue by the withdrawal of the panel-restricting tube 159 within the urethra 101.

FIG. 62 indicates the advancement of the arch 151 in the needle 152 into the recess 123 under the surface of the mucosa 113.

FIG. 63 depicts the deployment of the arch 151 beneath the surface of the mucosa 113 by withdrawing the needle 152 while holding the plunger 153 behind the arch 151 stationary.

FIG. 64 shows the retrieval of the resilient panel 122 by withdrawing the delivery device 107 into the panel-restricting tube 159, while the arch remains and elevates the mucosal 113 tissue.

FIG. 65 indicates a delivery device 107 with a built-in recess 123 and a receiving trough 160 open from the recess 123 to the distal end of the delivery device 107.

FIG. 66 shows a magnet 132 to be delivered into the urethral wall by the trocar 109 and delivery device.

FIG. 67 depicts a pair of magnets 132 attracting each other.

FIG. 68 shows a cross section of magnetic lumen 100 closure constricted by two attracting magnets 132 within anterior 118 and posterior 119 urethral walls.

FIG. 69 depicts the mid-longitudinal view of two magnets 132 and delivery devices 107, one above the other, facing opposite directions during urethral 101 insertion.

FIG. 70 indicates mutual packing of mucosa 113 into both recesses 123 of the delivery devices 107 as they align with each other. Both trocars 109 are in position to be inserted beneath mucosal 113 surfaces within the recesses 123.

FIG. 71 shows a swellable closure device 134 and a magnet closure device 132 with sutures 21 attached for ease of retrieval.

FIG. 72 depicts a swellable 134 and a magnetic 132 closure device with biodegradable coatings for direct penetration into the urethral wall.

FIG. 73 indicates a compressed urethra 101 and a restricted lumen 100 encroached upon by the enlarged prostate 124.

FIG. 74 shows four magnets 132 arranged in repelling polarities beneath the mucosa 113 to open or enlarge the lumen 100.

FIG. 75 depicts anchoring devices 149 and tissue ingrowth openings 150 on the magnet 132 to minimize device migration.

### DETAILED DESCRIPTION OF THE EMBODIMENTS

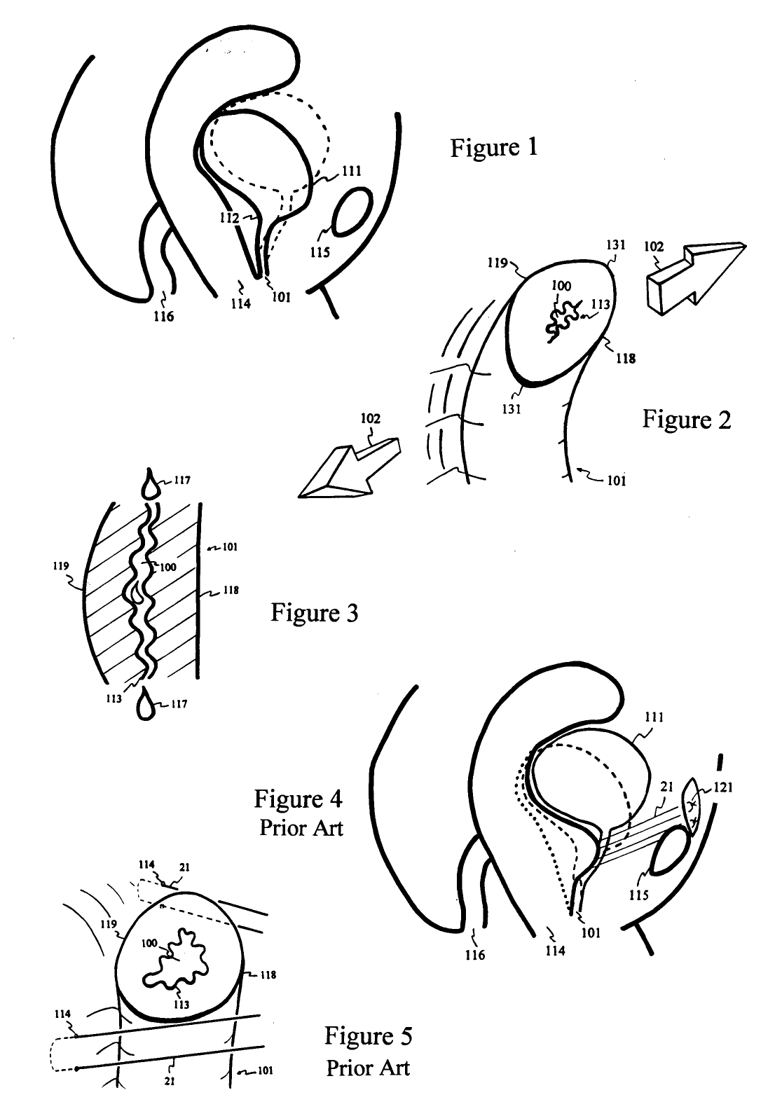

It is widely believed that most of the urinary incontinence in women is related to the descended position of the bladder 111, the funnneling of the bladder neck 112 and/or diminishing posterior 119 urethral support. The dashed line of FIG. 1 indicates the normal position and the solid line depicts the descended position of the bladder 111 with its funnel-shaped bladder neck 112. FIG. 2 shows a failed lumen 100 closure and hypermobility under stress with the urethropelvic ligament 102 pulling the lateral walls 131 of the poorly supported urethra 101. The mid-longitudinal view of FIG. 2 during stress is shown in FIG. 3, with urethropelvic ligaments pulling perpendicularly above and below the plane of the page. A section of poorly-supported posterior wall 119 withdraws from mucosal 113 coaptation, leading to urine 117 leakage.

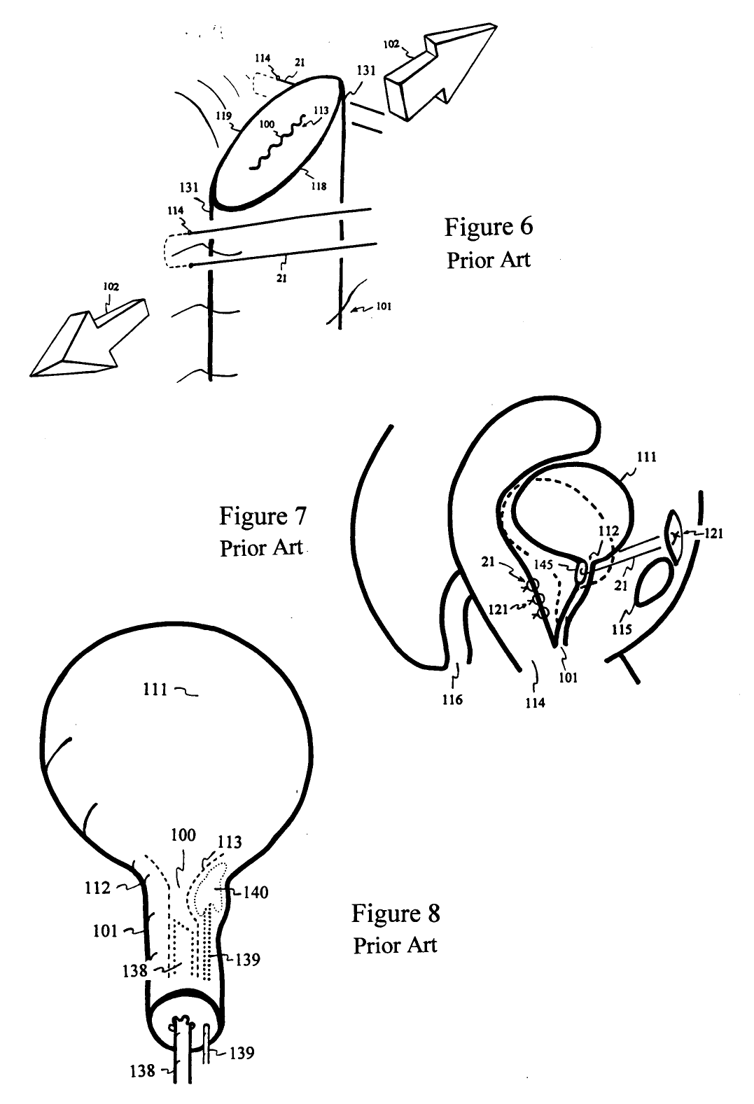

Numerous prior art surgical procedures are designed to treat urinary incontinence. The traditional surgical treatment for urinary incontinence is to add backboard support to the urethral posterior wall 119, usually by repositioning the vagina 114 with sutures 21. FIG. 4 indicates the pre-surgical position of the vagina 114 with a dotted line, and that of the urethra 101 and bladder with dashed lines. The post-surgical positions of the vagina 114 and vaginal backboard-supported urethra 101 are depicted with solid lines. FIG. 5 indicates a section of the vaginal backboard-supported posterior wall 119. This significantly invasive procedure provides the backboard support needed for urethral sphincteric closure during stress with concurrent pulling of the urethropelvic ligament 102, as shown in FIG. 6. The sling procedure is designed to loop a suture 21, tissue or other material behind the bladder neck 112 or urethra 101 to gently compress and restrict the lumen. FIG. 7 illustrates the sling correction from a pre-surgical position in dashed lines to a manageable opening at the bladder neck 112 in solid lines. Bulking agents 140, such as collagen, PTFE, silicon and fat, are injected beneath the mucosa 113 to narrow the lumen 100 while being monitored by an endoscope 138 as show in FIG. 8 using a periurethral injection technique. All of these prior art have significant shortcomings as mentioned in the background.

Several versions of rod-like or tube-like sphincteric closure devices are designed for implantation with a delivery device 107 beneath the surface of mucosa 113 to close the lumen 100 by elevating or lifting the mucosa 113. Unlike the artificial sphincters requiring manual operation, the rod-like or tube-like sphincteric closure devices allow the urethral 101 muscle to contract and initiate urination. In essence, the sphincteric closure devices specifically assist lumen 100 closure, while minimally interfering with the set of muscles responsible for voiding.

### Arch Closure Device

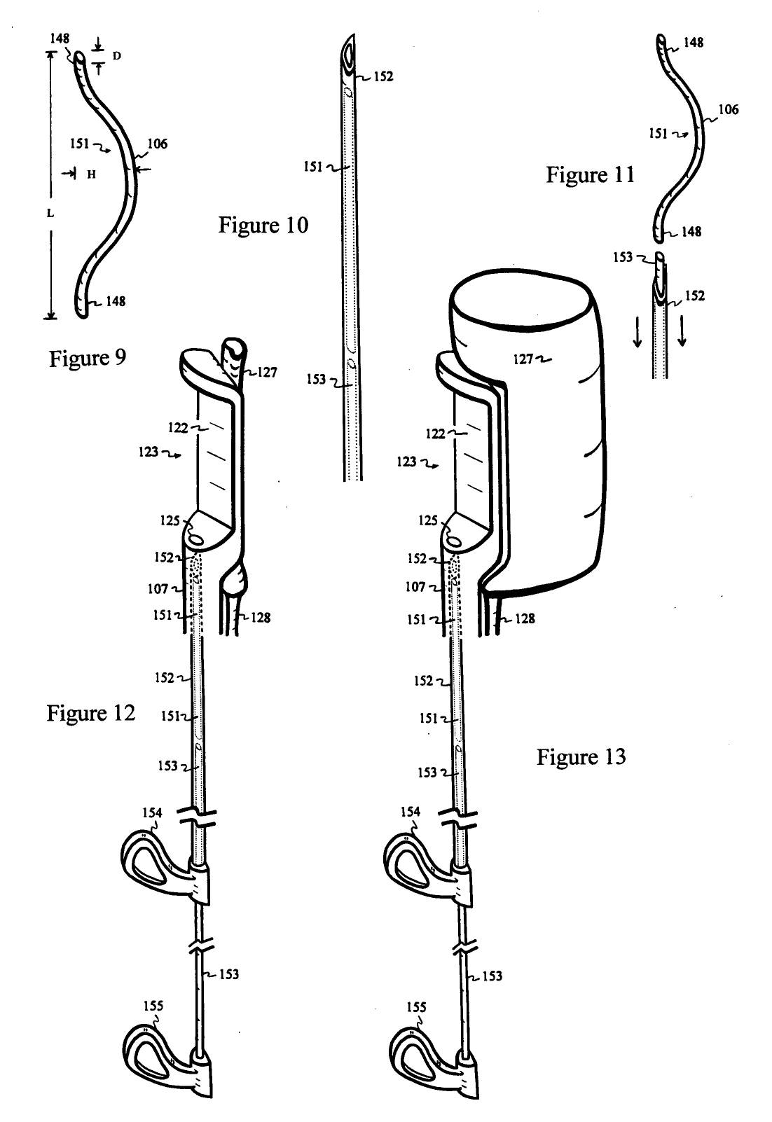

A resilient or elastic arch 151 with a curvature 106 and legs 148, shown in FIG. 9, is implanted beneath the mucosal 113 surface as a sphincteric closure device to shape and assist closure of the urethral lumen 100. The physical dimensions of the arch 151, as indicated in FIG. 9, are key elements in the sphincteric closure. The tightness and range of lumen closure are related to the elasticity of the arch 151 and the height, H, of the curvature 106. The length of sphincteric action is related to the length, L, of the arch 151, the base and shape of the curvature 106. The width of the sphincteric action is related to the diameter, D, of the arch 151.

For delivery, the arch 151 is resiliently straightened in a needle 152 with a plunger 153 behind the arch 151, as depicted in FIG. 10. The arch 151 is deployed to resume the pre-disposed curvature 106 by the withdrawal of the needle 152 while the plunger 153 is held stationary, as shown in FIG. 11. For lumen 100 closure, the direction of the resumed curvature 106 after deployment is crucial. The cross-sectional shape of the arch 151 and the passage in the needle 152 can be made non-round, elliptical for example, to avoid rotation of the resiliently straightened arch 151 within the needle 152 and to control the direction of deployment. The needle 152 and arch 151 assembly are loaded in a delivery device 107 with an indented pocket or recess 123 and a compressing balloon 127, as indicated in FIG. 12. The cross section of the delivery device 107 is shaped like a semi-circular rod with an inflatable balloon 127 behind the device 107. Both ends or walls of the indented recess 123 are preferred to be sloped and rounded for gentle insertion into and withdrawal from the urethra 101. For ease of urethral 101 insertion and withdrawal, the balloon 127 of the delivery device 107 is deflated. When the balloon 127 is inflated within the urethral wall, it pushes the adjacent recess 123 into the soft urethral 101 tissue. Due to the indentation, the mucosal 113 tissue is sheltered from full compression and is loosely packed or less flattened within the recess 123. The needle 152 carrying the arch 151 passes through an opening in the recess 123 wall, penetrating beneath the surface of the loosely packed mucosa 113 within the recess 123.

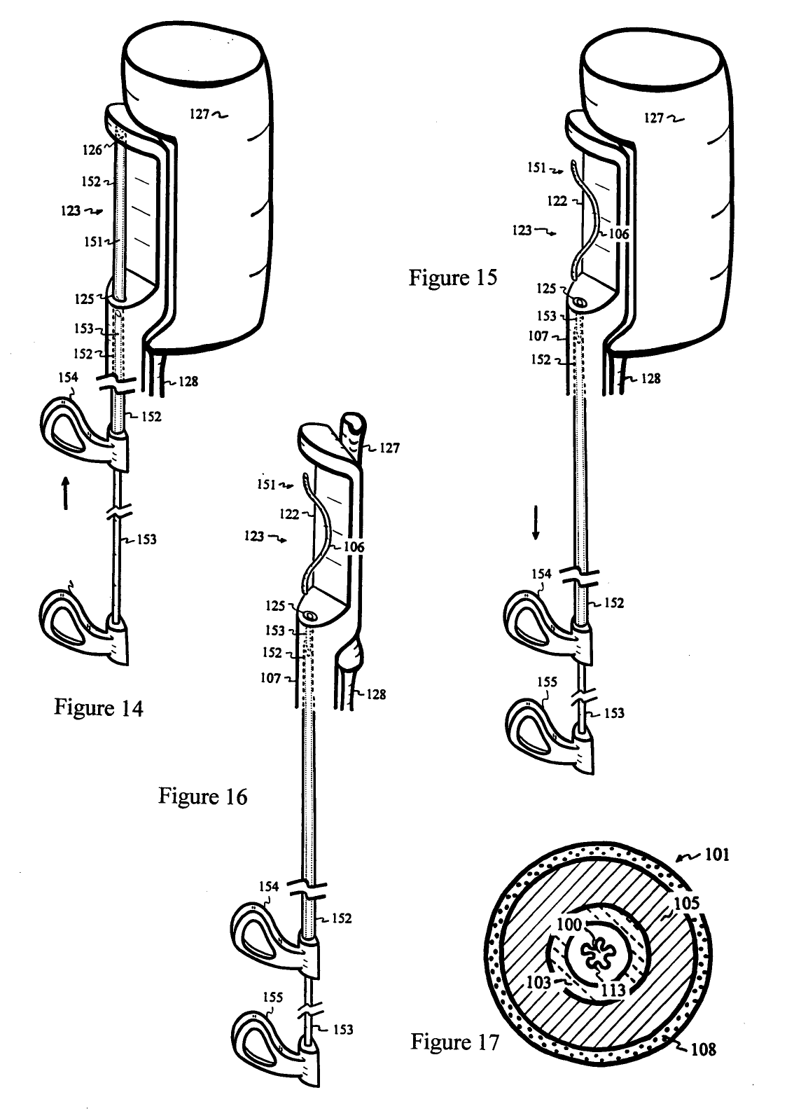

The implantation of the arch 151 involves the following simple steps. Insert the delivery device 107, as indicated in FIG. 12, into the urethra 101. Inflate the balloon 127, as shown in FIG. 13. Advance the needle 152 housing the arch 151 through the recess 123, as shown in FIG. 14. Deploy the arch 151 by withdrawing the needle 152 while holding the plunger 153 behind the arch 151 stationary, as indicated in FIG. 15. Deflate the balloon 127, as shown in FIG. 16. Withdraw the delivery device 107 from the urethra 101.

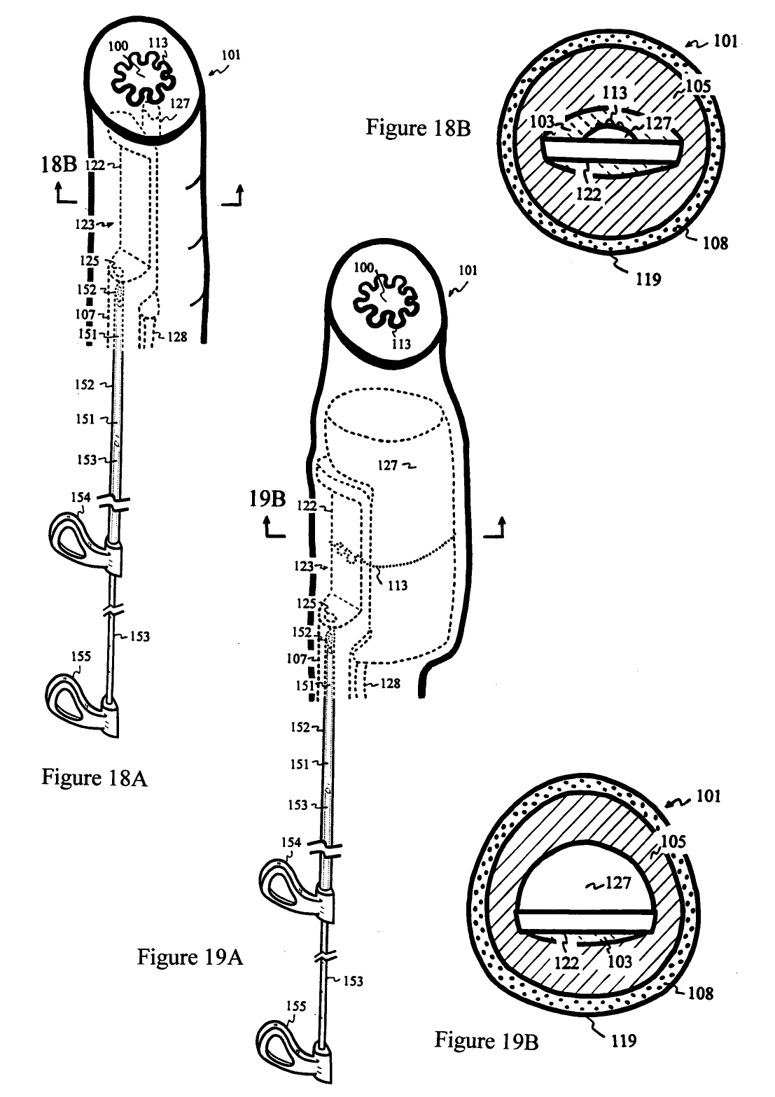

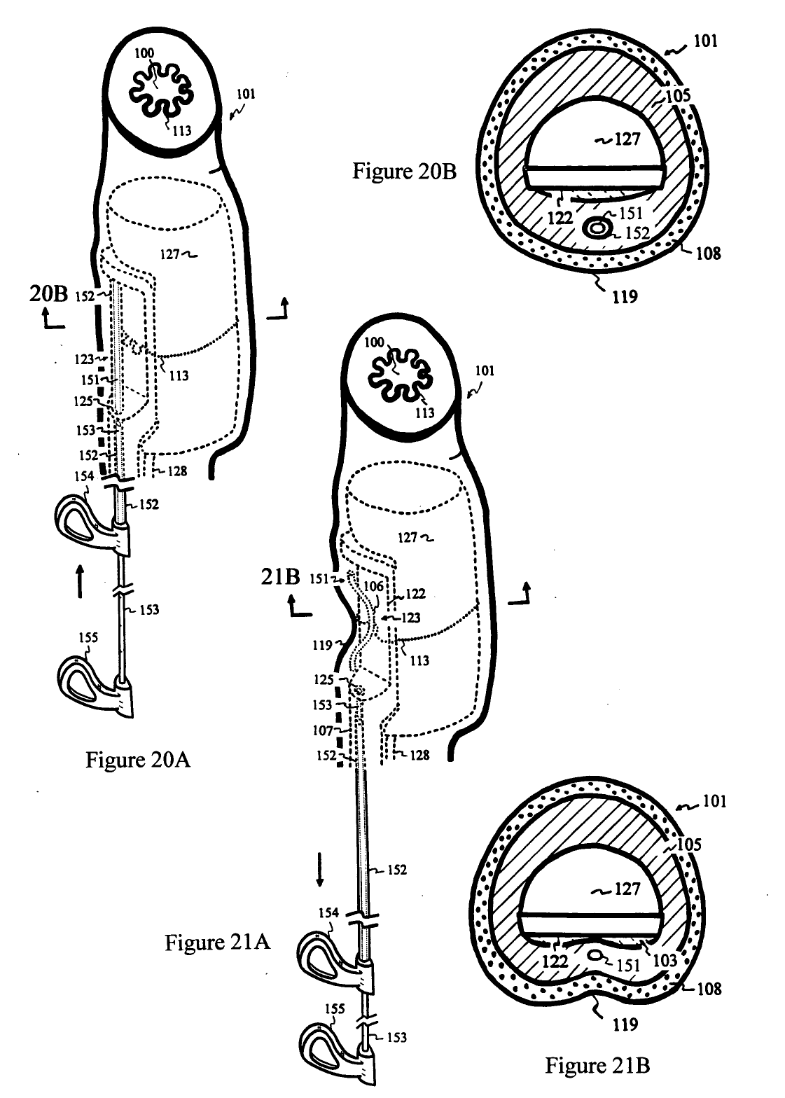

FIG. 17 depicts the cross section of a urethra 101 with sphincteric deficiency indicated by an open lumen 100 surrounded by mucosa 113, submucosa 103, smooth muscle 105 and striated muscle 108. The delivery device 107 is inserted into the sphincteric region of the urethra 101 as depicted in FIG. 18A, with a cross section marked 18B which is depicted in FIG. 18B. FIG. 18B shows the cross section of the balloon 127 prior to inflation and the panel 122 in the urethra 101. The balloon 127 is inflated, pushing or positioning the recess 123 against the urethral 101 wall. A plane of mucosa 113 is indicated by a dotted line around the recess 123, as shown in FIG. 19A. Due to the indentation, the mucosal 113 tissue within the recess 123 is loosely packed or less flattened, indicated by the ripple dotted line in front of the indented panel 122 of the recess 123, while the soft mucosal 113 tissue surrounding the recess 123 is compressed or flattened, also indicated in FIG. 19A. FIG. 19B depicts a cross section of loosely packed submucosa 103 against the indented panel 122, while the surrounding submucosa 103 is compressed and mostly hidden by the inflated balloon 127. Through a deployment opening 125, the needle 152 housing the arch 151 is pushed by the needle advancer 154, penetrating beneath the surface of mucosa 113, as indicated in FIG. 20A, into a receiving opening 126 shown in FIG. 14. A cross section of FIG. 20A is depicted in FIG. 20B, showing the arch 151 in the needle 152 puncturing through the smooth muscle 105 within the recessed region. Even if the needle 152 punctures a blood vessel in the submucosa 103 or smooth muscle 105, tissue compression by the inflated balloon 127 would greatly minimize bleeding or hematoma.

Although the inflated balloon 127 provides posterior support to the device, the recess 123 portion of the delivery device 107 is preferably made with a rigid material to ensure the needle 152 is properly advanced from the deployment opening 125 to the receiving opening 126, while the remaining portion is constructed with flexible material to accommodate the curvature of the urethra 101. To prevent the needle 152 from puncturing the external wall of the urethra 101, the sharpened tip of the needle 152 is beveled or tapered from the external side to the indented panel 122, as shown in dotted lines in FIGS. 12, 13 and 19A. The depth of mucosal 113 penetration by the needle 152 is related to the depth of indentation of the recess 123, adjustable by the size and/or pressure of the inflated balloon 127. The thickness of the mucosa 113, submucosa 103, and the elasticity of the urethral tissue may also influence the depth of mucosal 113 penetration by the needle 152.

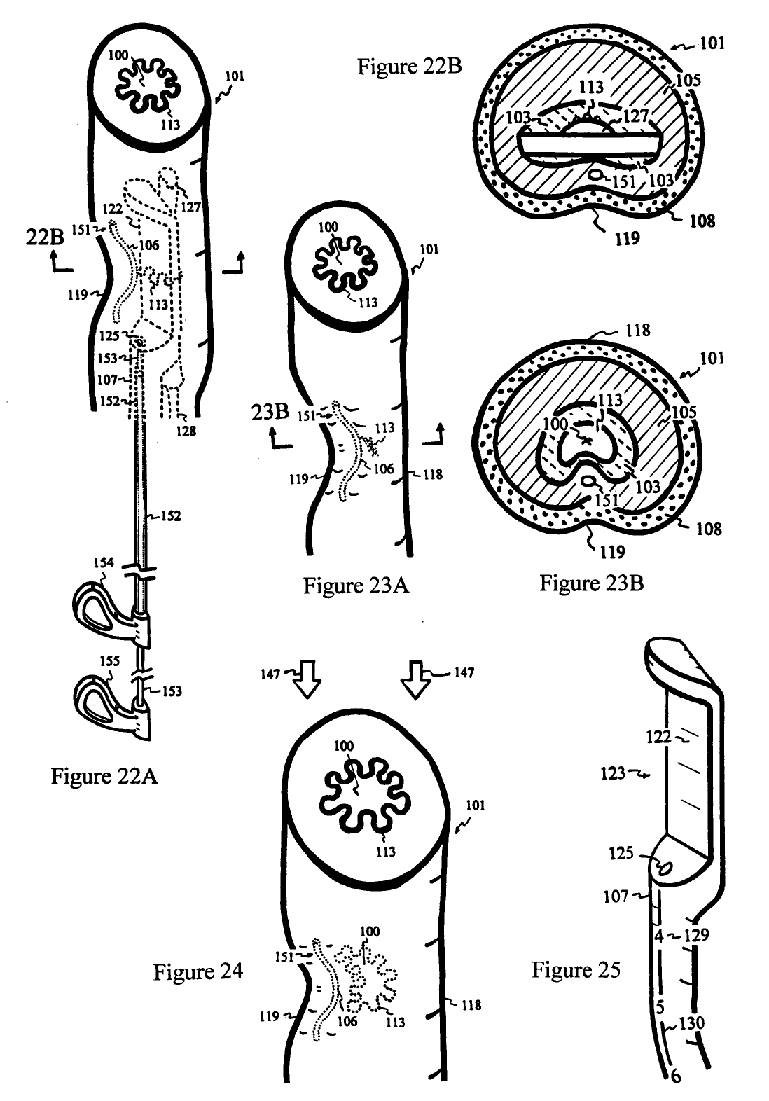

The arch 151 is then deployed beneath the surface of the mucosa 113 by pulling the needle advancer 154 while holding the plunger holder 155 stationary behind the arch 151, as depicted in FIG. 21A. As the restriction of the needle 152 is released, the arch 151 resumes the pre-disposed curvature 106 beneath the soft urethral 101 tissue, lifting the mucosa 113 toward the lumen 100, as indicated in FIG. 21A. A cross section of FIG. 21A is shown in FIG. 21B, indicating an elliptical arch 151 lifting smooth muscle 105 and submucosa 103 toward the indented panel 122 of the delivery device. The balloon 127 is then deflated and the deployed arch 151 is separated from the delivery device 107, as shown in FIG. 22A. FIG. 22B indicates a cross-sectional view of the deflated balloon 127 and the deployed arch 151 lifting the smooth muscle 105 and submucosa 103 inward. After the delivery device 107 is withdrawn from the urethra 101, the curvature 106 of the elastic arch 151 elevates the supple mucosa 113, narrowing or closing the previously opened lumen 100, as depicted in FIG. 23A. FIG. 23B shows a cross section of the urethra 101 with a closed lumen 100 resulting from elevation of the mucosa 113 by the arch 151.

For stress closure of the lumen 100, the arch 151 is preferably implanted within the muscle-poor posterior wall 119 to elevate it towards the muscle-rich anterior 118 mucosa 113 for coaptation and closure, as shown in FIG. 23A. During stress, such as coughing or sneezing, the urethropelvic ligaments 102 pull on the lateral urethral walls 131 to close the lumen 100. The arch 151 residing within the posterior wall 119 is unlikely to impede stress closure and probably promotes closure by adding backboard support.

Muscle distribution in the urethra 101 plays a crucial role in sphincteric control, both in closure and voiding. It is likely that voluntary urethral shortening and widening resulting in lumen 100 opening are mainly due to the muscular contraction of the muscle-rich anterior 118 urethral wall and partly by lateral 131 walls. The muscle-poor posterior 119 wall plays only a passive role with minor movement, which would not be significantly affected or irritated by the residing arch 151 during urethral 101 shortening or lengthening for initiating or interrupting urine flow. In fact, injection of bulking agent 140, as indicated in FIG. 8, requires certain injection locations within the urethra 101 in order to take advantage of the muscular distribution in the urethra 101 to gain control of closure and to allow natural voiding.

To treat sphincteric deficiency, injections are selectively filled at the 4 and 8 o'clock positions of the urethral 101 wall, where the urethral anterior 118 wall is at the 12 o'clock position. The bulging agent 140 overlaps at 6 o'clock, the posterior 119 wall position, lifting the posterior 119 half of the mucosa 113 to narrow or close the lumen 100. Some physicians inject directly into the posterior wall 119, the 6 o'clock position. To initiate voiding, the muscle-rich anterior wall 118 stiffens and shortens the urethra 101; at the same time the anterior wall 118 pulls forward, away from the bulging posterior wall 119 to allow the urine 117 to flow through. Similarly, to initiate voiding with an implanted arch 151 in the posterior wall, the anterior wall 118 stiffens and pulls beyond the closing, curving or lifting range of the arch 151. In essence, to initiate urination, the contraction of the anterior 118 wall widens the lumen 100 beyond the closing range of the arch 151, as depicted in FIG. 24, to allow urine 117 to flow through. After voiding, the muscles relax and the anterior 118 mucosal 113 wall returns within range again to be coapted with the posterior 119 mucosal 113 wall shaped by the arch 104 beneath to prevent unwanted urine leakage.

The depth of the arch 151 delivery is unlike the poorly controlled injection of bulking agent 140 beneath the mucosa 113 as depicted in FIG. 8. The amount of bulking agent 140 needed to be visible by the endoscope 138 unpredictably varies from 2 cc to 45 cc. It is quite possible that if the injection is just below the surface of the mucosa 113, only a small amount of agent 140 is needed to narrow the lumen 100. However, if the needle 139 is injected deep into the smooth muscle 105 or even into the striated muscle 108, the outer layer of urethra 101, a large amount of the agent 140 is likely required to narrow the lumen 100. The poorly controlled injection technique may be the cause of the unpredictable efficacy, durability, migration and/or immunogenicity of these bulking agents 140. On the other hand, the depth of arch 151 delivery is controlled by the pre-determined depth of the recess 123 and the size and/or pressure of the balloon 127. The sphincteric region is estimated by the penetration markers 129 labeled on the delivery device 107 as shown in FIG. 25; and the direction of curvature 106 is aligned with the orientation line 130, also shown in FIG. 25, to reproducibly implant a durable and non-immunogenic sphincteric closure arch 151.

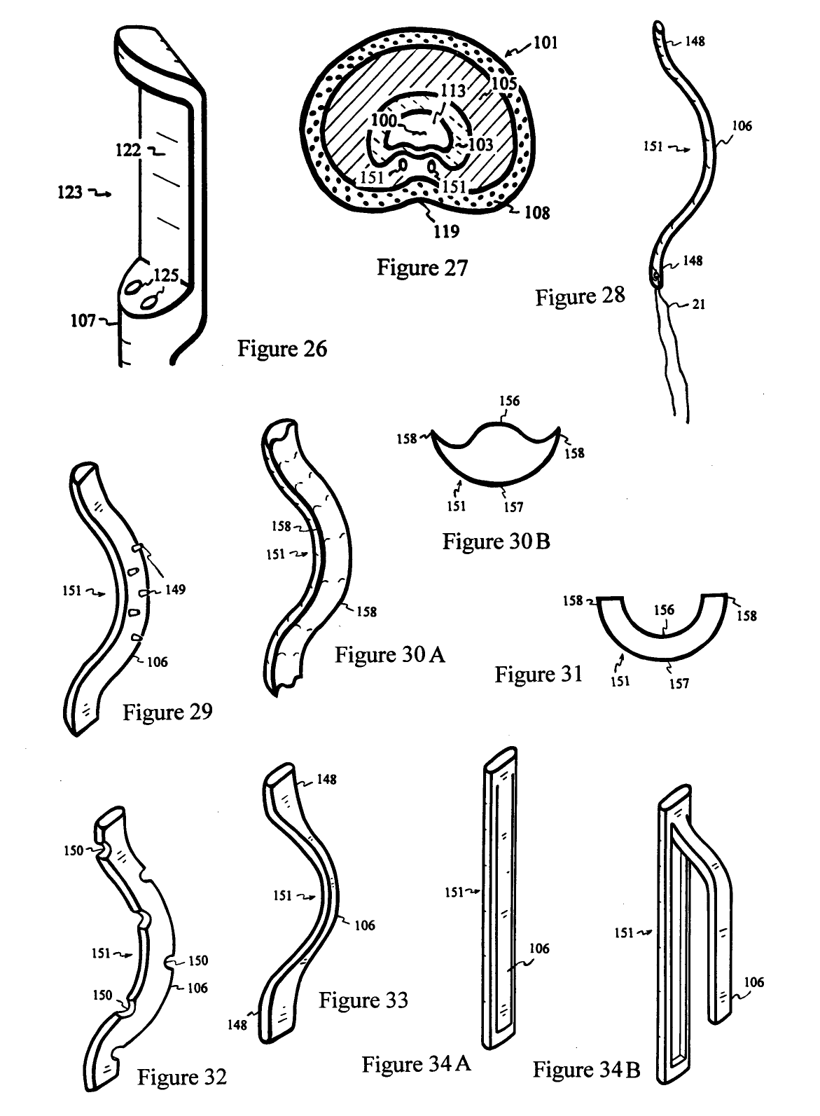

Increasing the diameter of the arch 151 may improve lumen 100 closure by elevating a larger segment of the mucosa 113. Likewise, delivering two arches 151, from a double needle delivery device 107 equipped with two deployment openings 125, as shown in FIG. 26, may further improve lumen 100 closure. The cross-sectional view in FIG. 27 shows the result using double arches 151 to lift a wide section of mucosa 113 and to fill the lumen 100.

In an event requiring arch 151 removal, such as infection, non-performance, pain or urine retention, a suture 21 attached to the arch 151 as depicted in FIG. 28 can be used as a retrieval device. The suture 21 is preferred to be made with degradable material. The suture 21 linked arch 151 is implanted as mentioned, with a portion of the suture 21 leading to the lumen of the urethra 101. In the event that arch 151 retrieval is necessary, the suture 21 is pulled to retrieve the arch 151 from the soft and supple urethral 101 tissue. Otherwise, the degradable suture 21 will be cut, voided and/or metabolized.

The long-term stability of the implanted arch 151 is attributable to the design of the arch 151 and the opened space or vacancy created by the lumen 100. FIG. 23A shows a curved arch 151 with legs upon which the curvature 106 pivots. FIG. 23B, cross-sectional view of FIG. 23A, indicates one leg 148 of the arch 151 pointing above while the other leg 148 points below the page, pivoting the curved portion to lift the soft submucosa 103 and mucosa 113 toward a vacant space created by the previously open lumen 100. In essence, the curvature 106 is the pressure point with slight pivotal movement provided by the legs 148, pressing into an indentation, securely nesting, filling, narrowing and/or closing the vacant gap of the lumen 100. The compression of the curvature 106 forms a valley or a pocket of soft urethral 101 tissue, indented toward the direction of the lumen 100, as depicted in FIG. 23B. Once the valley of soft urethral 101 tissue is embedded around the curvature 106, the shifting of the curvature 106 from facing the lumen 100 to sideways should be minimal. Furthermore, the curvature 106 is likely to favor settling into the most supple tissues in the urethra 101, such as the mucosa 113 and submucosa 103, which would direct the curvature 106 toward the lumen 100. Therefore, the direction of the curvature 106 toward the lumen 100 is likely to be the most stable configuration. During voiding, the anterior 118 wall pulls away from the curvature 106, magnifying the vacant lumen 100, creating a deepened indentation, which further secures the direction and stability of the sphincteric closure device.

It is also possible to further secure the arch 151 within the urethral 101 wall by additional arch 151 designs. The protruded anchoring devices 149, as depicted in FIG. 29, prevent pivotal swinging of the curvature 106 from facing the lumen 100 to shifting to the side, as well as arch 151 migration. To minimize pivotal swinging of the curvature 106, the lateral 158 walls of the arch 151 can also be shaped to anchor urethral tissue, as shown in FIG. 30A. The cross section of FIG. 30A shows the lateral 158 walls for tissue anchoring and the anterior 156 wall for mucosa 113 lifting. The anterior 156 portion of the arch 151, especially around the pressure point of the curvature 106, can also be used to anchor urethral 101 tissue to prevent pivotal swinging. FIG. 31 depicts a cross section of a curvature 106 of an arch 151, with an indentation in the anterior 156 side to hold and anchor urethral 101 tissue and to prevent pivotal swinging of the curvature 106. To minimize device migration with time, tissue ingrowth openings 150 on the sphincteric closure device, as indicated in FIG. 32, can be helpful especially in tissue with movement, such as the urethra 101. The pivotal swinging of the curvature 106 can also be significantly reduced by having a narrow curvature 106 supported with wide legs 148, as depicted in FIG. 33.

Due to the supple texture of the urethra 101, urine retention from excessive lumen 100 closure or urethral 101 kinking is one of the most common complications in current surgical procedures. An arch 151 is specifically designed to narrow or close the lumen 100 with minimal risk of urethral 101 kinking. The arch 151 is also made with elastic or shape memory material, and a curvature 106 capable of resiliently flattening within the body of the arch 151, as shown in FIG. 34A. As the restriction on the curvature 106 is lifted, the pre-disposed shape of the curvature 106 protrudes out from the body of the arch 151, as depicted in FIG. 34B. The arch 151 can be delivered by the delivery device 107 beneath the surface of mucosa 113 into the urethral 101 wall. The unique function of the arch 151 in FIG. 34B is to thicken the soft urethral 101 tissue, narrowing or closing the lumen 100 by lifting the mucosal 113 layer to improve coaptation. The body or the base of the arch 151 also adds support to the urethral 101 wall, eliminating the possibility of urethral 101 kinking.

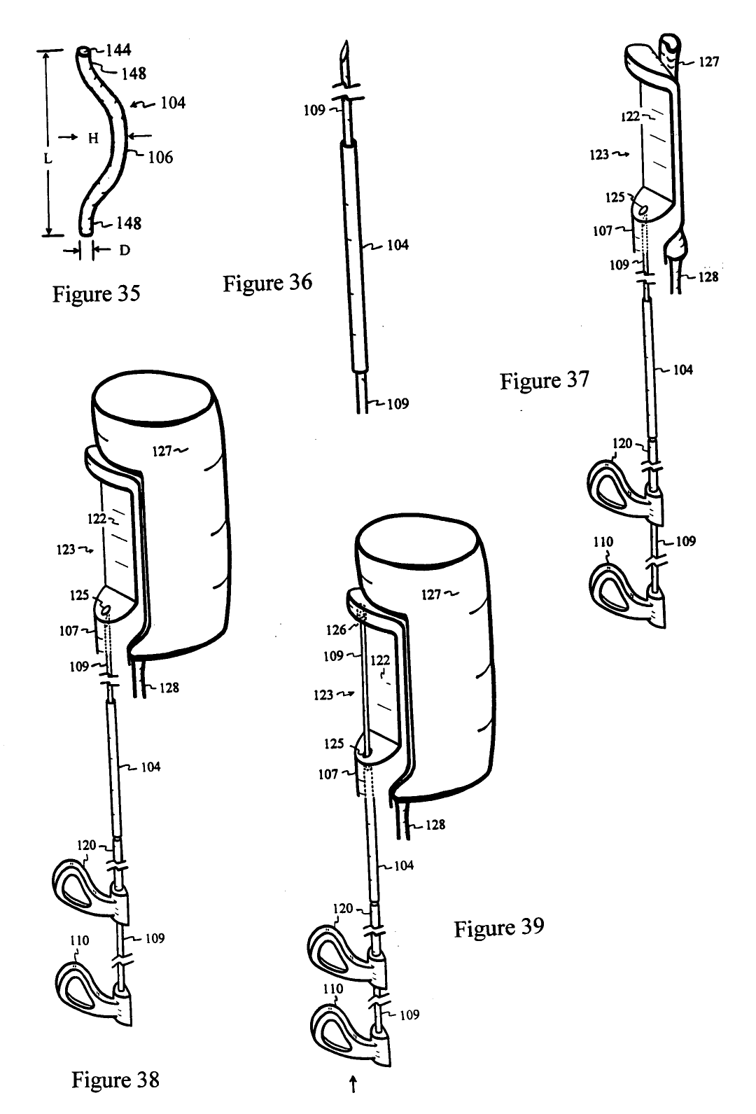

Instead of delivering the sphincteric closure device with the needle 152, an arch tube 104 with a passage 144, legs 148 and curvature 106, as shown in FIG. 35, is designed to be delivered by a trocar 109. The arch tube 104 is also made with elastic or shape memory material, capable of being resiliently straightened by a relatively rigid trocar 109 sized and configured to fit into the passage 144 of the arch tube, as depicted in FIG. 36. To prevent rotation of the arch tube 104 around the trocar 109, the passage 144 and the cross-sectional shape of the trocar 109 can be made non-round or elliptical.

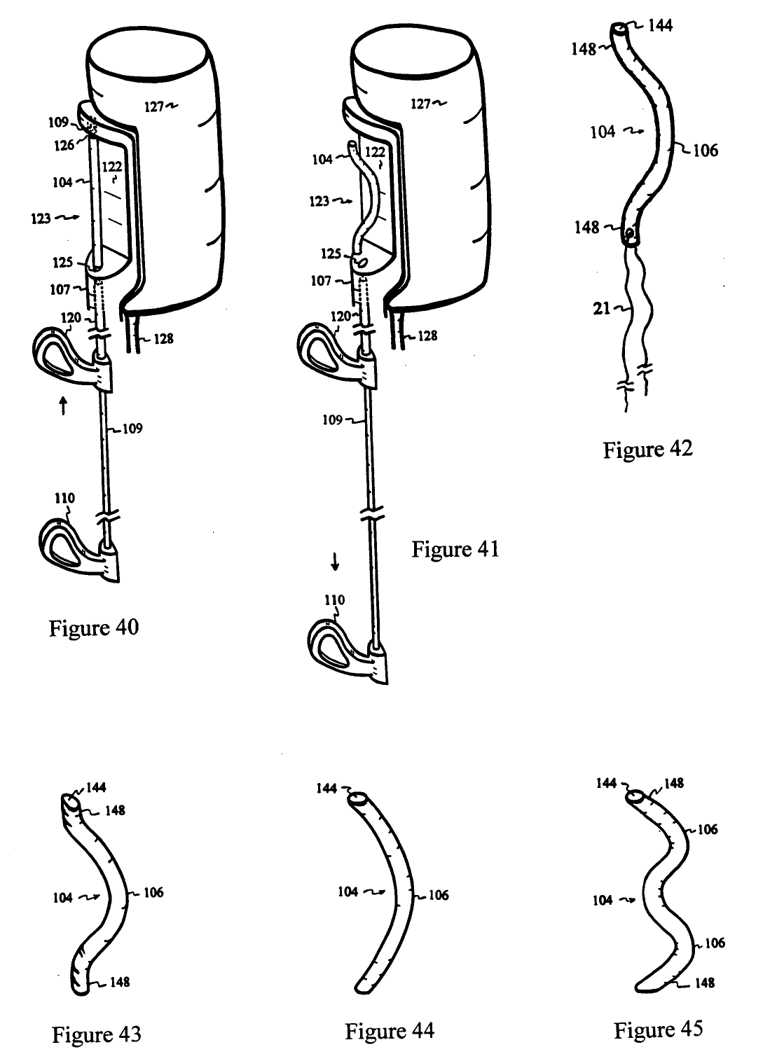

The implantation of the arch tube 104 involves only the following simple steps: (1) insert the delivery device 107 containing the trocar 109 and the arch tube 104, as indicated in FIG. 37, into the urethra 101, (2) inflate the balloon 127, as shown in FIG. 38, to press the recess 123 against the urethral tissue, (3) advance the trocar 109 through the recess 123 beneath the surface of the mucosal tissue by pushing the trocar advancer 110, as shown in FIG. 39, (4) slide the arch tube 104 over the trocar 109 by pushing a device advancer 120, as indicated in FIG. 40, and (5) deploy the arch tube 104 within the urethral wall by withdrawing the trocar 109 while holding the device advancer 120 stationary, as depicted in FIG. 41, (6) deflate the balloon 127, and (7) withdraw the delivery device 107 from the urethra 101. The deployed arch tube 104 is implanted in the urethral 101 wall, elevating the mucosa 113 to close the lumen 100. A suture 21 attached arch tube 104 is shown in FIG. 42. During adverse events, the arch tube 104 can be easily retrieved by pulling on the suture 21.

The length, width, elasticity, height and shape of the curvature 106 of the arch 151 body or arch tube 104 body play essential roles in the closure of the lumen 100 at rest and in the widening of the lumen 100 during voiding. The length, L, of the arch 151, 104, as shown in FIGS. 9 and 35, should be less than 5 cm, preferably about 1 to 3 cm occupying the sphincteric region of the urethra 101. The outer diameter, D, should be less than 5 mm, preferably about 0.2 to 2 mm to elevate mucosa 113 and to close the lumen 100. The height, H, of the curvature 106 should be less than 5.5 mm, and more likely less than 3.5 mm.

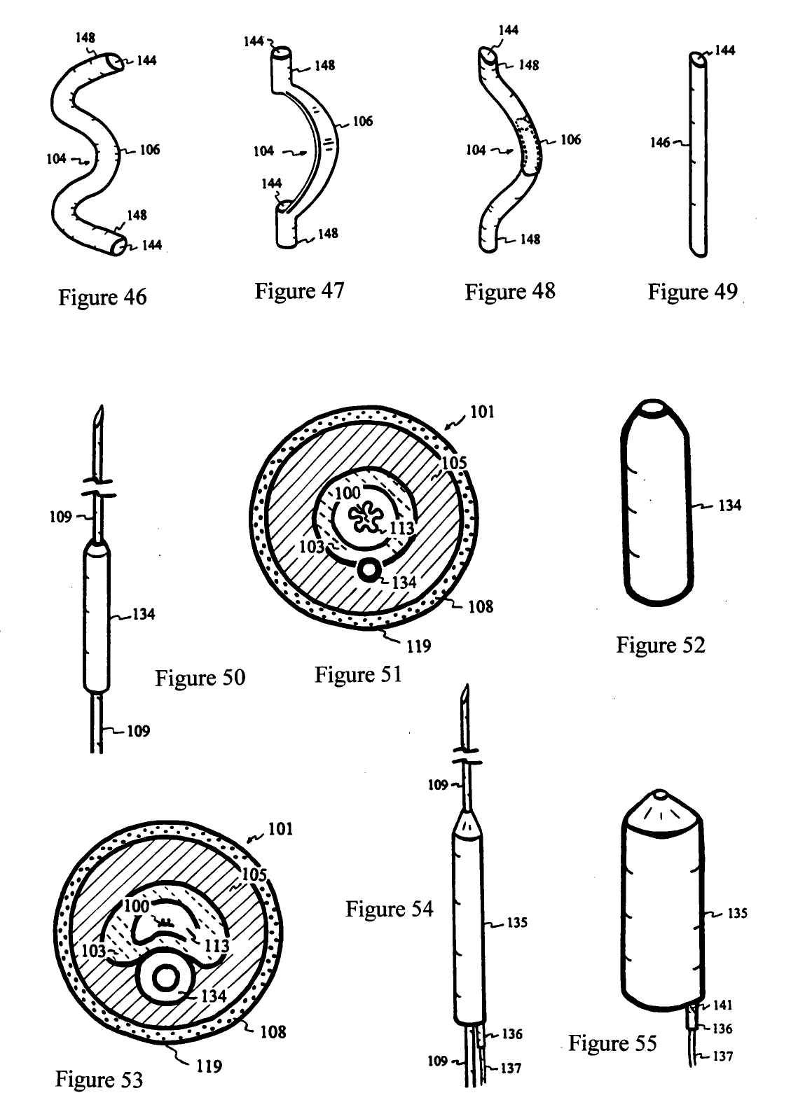

The shape and configurations of the curvature 106 or the pressure points also contribute to sphincteric efficiency. The flat side or the wide side of the elliptical arch tube 104 in FIG. 43 is curved toward the lumen 100 to increase the surface area for elevation. The curvature 106 can be lengthened by shortening or eliminating the legs, as indicated in 44, to increase the length of the sphincteric closure and to decrease the pivotal movement of the curvature 106. It is also possible to have double curvatures 106, as depicted in FIG. 45, to double the convex compression and double the sphincteric actions. The concave side of the arch 151 or 104 can also be oriented toward the lumen 100 to provide compression by the bases of the curvature 106 to close the lumen 100. Furthermore, by involving both ends or the legs 148, with the curvature 106, as indicated in FIG. 46, three pressure points generate three sphincteric actions within a small section of the sphincteric region. The stiffness of the curvature 106 can be greatly reduced by connecting two tubular legs 148 to a narrow curved 106 strip, as shown in FIG. 47, for gentle lumen 100 closure. Furthermore, the height, H, of the curved 106 strip can be adjusted by the spread of the legs 148, illustrated in FIG. 47. The arch tube 104 can also be composed of modular components to provide selective physical properties, dimension, biodegradative profile, tissue ingrowth, movement or other added benefits. FIG. 48 shows a two-pieced arch tube 104, designed to accommodate potential shortening and lengthening movements at the implant site.

Especially for Types 0, I, IIA and IIB incontinence, merely supporting the posterior 119 urethral wall might be sufficient to correct the incontinent problem. To support the urethral wall, a tube or a rod with adequate rigidity as a backboard support, indicated in FIG. 49, can be micro-invasively delivered with the delivery device 107 into the posterior 119 wall, to minimize the withdrawal of the posterior 119 wall during stress.

The muscle-poor posterior urethral wall 119 is the preferred site for implanting the sphincteric closure devices for the following reasons: (1) it provides the backboard support to further gain sphincteric control, (2) the location does not impede stress lumen 100 closure initiated by the tension of urethropelvic ligament 102, and (3) it frees the muscle-rich anterior wall 118 to widen the lumen 100 to urinate.

Biocompatibility and elasticity of the arch 151 or arch tube 104 are both important to ensure long term success. Nickel titanium, nitinol, has been used in urethral stents with no evidence of foreign body reactions or corrosion (D. Latal et. al., Urol. Res., 22: 295-300, 1994). The super-elastic property of nickel titanium will allow a curved arch to be resiliently straightened by the needle 152 or the trocar 109. The shape memory property of the nickel titanium can also provide both curvature and straightening by temperature alteration. Other alloys, such as a stainless steel tempered spring may have the elastic characteristics. Some polymers, such as polypropylene, polyethylene, polyurethane, poly-ether-ketone, DELRIN (acetal resin), polysulfone, polycarbonate, polyimide, polytetrafluoroethylene or others may also be biocompatible and have the elastic modulus to tolerate straightening and mucosal 113 shaping. Especially for testing purposes, a biodegradable arch 151, 104 can be made with poly-lactate, poly-glycolic or other biodegradable materials. All material should be able to withstand sterilization by gamma, electron beam, ETO, steam or other technique to prevent infection.

To improve the performance and/or visibility, the arch 151 or arch tube 104 can be coated with antibiotic, hormone, growth factor, analgesic, blood clotting, nutrient, radiopaque, echogenic, lubricant, swelling, plasma coating and/or other materials.

### Swellable Closure Device

Instead of closing the lumen 100 by a curvature 106 of the arch 151, 104, a swellable closure device 134 can also be delivered with the trocar 109, as indicated in FIG. 50, through a similar delivery device 107. The swellable closure device 134 is implanted beneath the surface of the mucosa 113, as depicted in the cross-sectional view in FIG. 51, by similar procedures operating the delivery device 107, as shown in FIGS. 38 to 41. After absorbing water or serum, the closure device 134 swells and greatly increases in diameter and possibly in length, as indicated in 52. The swellable closure device 134 hydrates and swells within the urethral 101 tissue, pushing the mucosa 113 in toward the vacant space of the opened lumen 100 to narrow and/or close the lumen 100, as depicted in a urethral 101 cross section in FIG. 53.

Collagen, hyaluronate and their derivatives can be processed, dried and sterilized to form a swellable closure device 134. Hydrophilic polymers, such as polyethylene glycol can be crosslinked, shaped, dried and sterilized to form a swellable closure device 134. To improve performance and/or visibility, the swellable device 134 can also be coated or combined with antibiotic, hormone, growth factor, analgesic, blood clotting, nutrient, radiopaque, echogenic, lubricant, and/or other materials.

### Inflatable Closure Device

Instead of relying upon swelling to induce lumen 100 closure, an inflatable closure device 135, as shown in FIG. 54, can be folded or rolled tightly in the delivery device 107 to be delivered into the urethral 101 wall. The inflated closure device 135, depicted in FIG. 55, is equipped with a one-way valve 141 in a stem 136 connected to a detachable tube 137 for inflating a chamber of the device 135. The implantation of the inflatable closure device 135 beneath the mucosa 113 is very similar to the method of delivering the swellable closure device 134, as indicated in FIG. 51. The result of lumen 100 closure is also very similar to the result using the swollen closure device 134, as depicted in FIG. 53. However, one advantage of using the inflatable closure device 135 is that the lumen 100 closure can be adjusted by the amount or the pressure of inflating medium, while the swellable closure device 134 depends on the diameter, depth of implantation and swelling capability. The bag of the inflatable closure device 135 can be made of silicone or polyurethane and inflated with a small amount of inert, viscoelastic and long lasting silicone oil. The inflating medium can also be air, gas or water. To achieve proper lumen 100 closure, the amount of the medium can be monitored by pressure, volume and/or endoscopic observation, as in monitoring bulking agent 140 injections in FIG. 8.

Similar to the implanted arch 151, 104, the swollen 134 or inflated 135 device also presses into the indentation of the vacant space created by the opened lumen 100, securely resting within the preferred posterior 119 urethral 101 wall. To initiate voiding, detrusor contraction 147 pushes from the abdomen; the anterior 118 urethral wall stiffens and pulls away from the muscle-poor posterior 119 wall, exceeding the closing range of the swollen 134 or inflated 135 device to enlarge the lumen 100 and to urinate. After voiding, the muscles relax and the anterior 118 mucosal 113 wall is once again within range to be coapted with the posterior 119 mucosal 113 wall, shaped by the swollen 134 or inflated 135 device, to close the lumen 100 and prevent leakage.

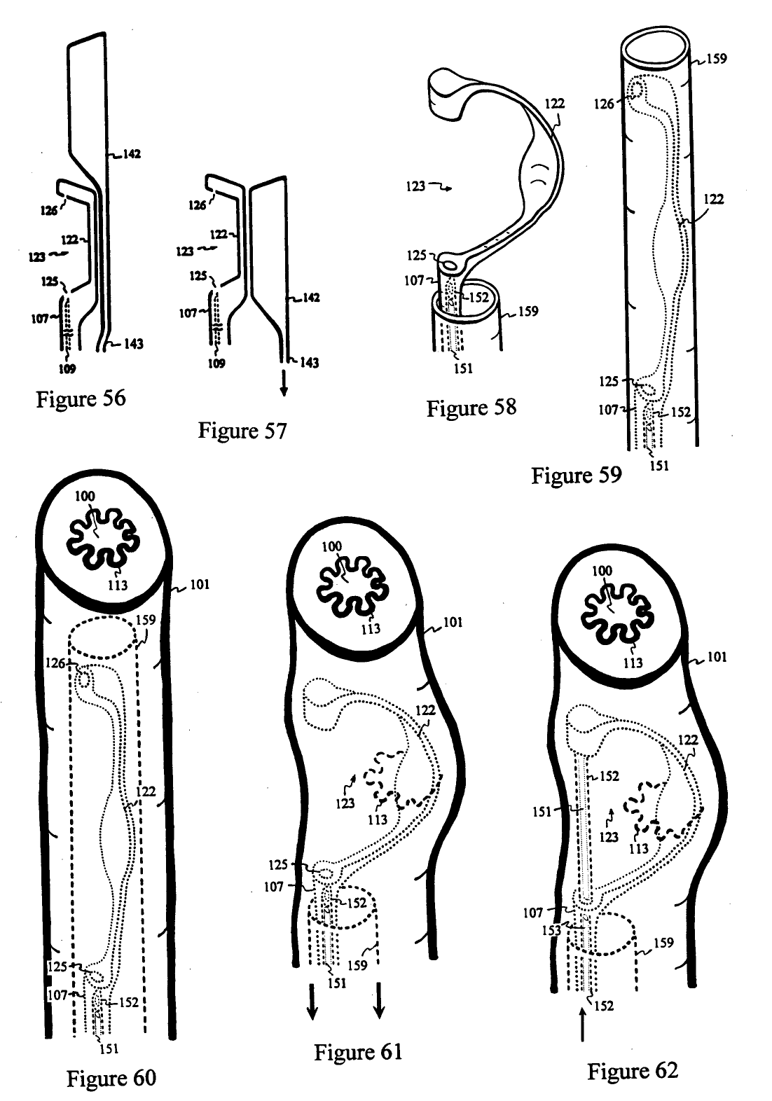

The delivery device 107 utilizes a balloon 127 to press or position the recess 123 into the supple mucosa 113 and other soft urethral tissue, allowing the needle 152 or trocar 109 to penetrate beneath the surface of the mucosal 113 layer. The balloon 127 can be replaced by a protruded recess positioner 142, placed above or below the recess 123 region of the delivery device 107, as shown in a mid-longitudinal view in FIG. 56, ready to be delivered into the urethra 101. The distal and proximal walls of the protruded recess positioner 142 are sloped, as indicated in FIG. 56, for ease of gliding and positioning with the recess 123 of the delivery device 107. Compression of the recess 123 into the supple mucosa 113 is achieved by pulling or manipulating the recess positioner handle 143 to align the protruded section of the positioner 142 with the recess 123 region, as shown in FIG. 57.

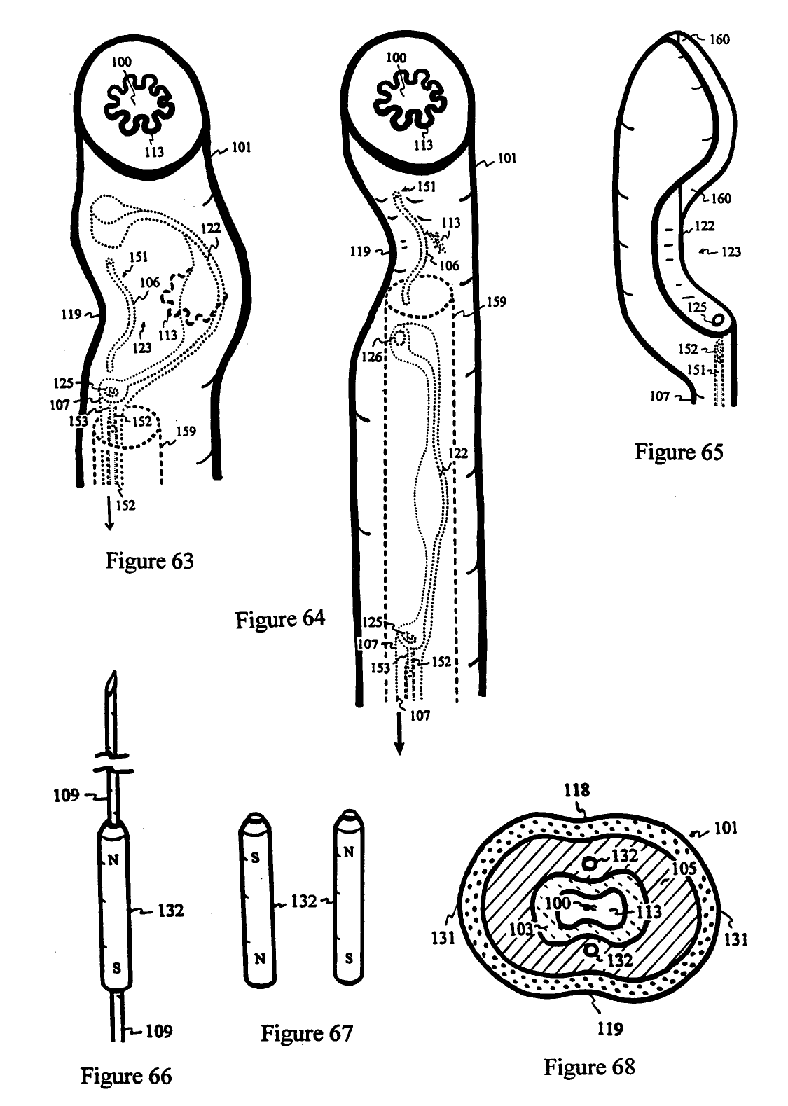

The panel 122 of the delivery device 107 can be made with elastic or shape memory material, forming a curvature to create a recess 123, as shown in FIG. 58, without the balloon 127 or the recess positioner 142. The elastic panel 122 can be retrieved and resiliently straightened into a panel-restricting tube 159, as shown in FIG. 59. The delivery device 107 with the resiliently straightened panel 122 in a panel-restricting tube 159 is inserted into the urethra 101, as indicated in FIG. 60. The elastic panel 122 resumes the pre-disposed curvature by withdrawing the panel-restricting tube 159. This causes both ends of the recess 123, the deployment 125 and receiving 126 openings, to be pressed beneath the surface of the mucosa 113, as depicted in FIG. 61. The arch 151 in the needle 152 is advanced through the recess 123, tunneling beneath the surface of the mucosa 113, as indicated in FIG. 62. The arch 151 is deployed by withdrawing the needle 152 from the recess 123 while holding the plunger 153 behind the arch 151 stationary as shown in FIG. 63. The panel 122 is then retrieved and resiliently straightened in the panel-restricting tube 159 by withdrawing the delivery device 107 while holding the panel-restricting tube 159 stationary, as depicted in FIG. 64. To minimize the possibility of scraping or dislocating the deployed arch 151 during the retrieval of the elastic panel 122, a groove can be indented at the receiving opening 126 to allow the arch 151 to slip through. It is also possible to rotate the delivery device 107 before retrieving the panel 122 into the panel-restricting tube 159 to minimize scraping the deployed arch 151. As indicated in FIG. 64, the deployed arch 151 remains in the urethral 101 wall, lifting the mucosal 113 tissue and closing the lumen 100.

The resilient panel 122 can be made with nickel titanium alloy for the elastic and/or temperature controlled shape memory capability. Other resilient material, such as spring tempered stainless steel, polypropylene, polyethylene, polyurethane or other polymer, may also be suitable.

Since the urethra 101 is elastic and can be lubricated, it may be feasible to insert a delivery device 107 with a built-in recess 123, as shown in FIG. 65, into the urethra 101. The distal portion of the delivery device 107 is enlarged to provide the recess 123. The cross section of the enlarged distal portion of the delivery device 107 can be made elliptical with the deployment opening 125 located at one of the elongated sides, as depicted in FIG. 65. During urethral 101 insertion, the urethra 101 conforms to the built-in recess 123, changing the urethral cross section from a round to an elliptical configuration, filling the recess 123 with mucosa 113 without causing excessive discomfort from circumferential expansion of the urethra 101. The delivery device 107 also contains a receiving trough 160 channeled from the recess 123 to the distal end of the delivery device 107, as shown in FIG. 65. The receiving trough 160 can serve as a needle 152 passage and also provide a channel or passage for the arch 151 to minimize scraping or dislocation of the deployed device within the urethral 101 wall during the withdrawal of the delivery device 107. A similar receiving trough 160 can be used in the delivery device 107, shown in FIGS. 18A to 22A, 38 to 41 and 58 to 64, to minimize contact with the deployed lumen closure device during retrieval of the delivery device 107. The recess 123 of the delivery device 107 can also be created by other means.

### Magnetic Closure Device

The outer diameter of the urethra 101 is usually less than 6 mm; the diameters of smooth muscle 105, submucosa 103 or mucosa 113 are even smaller. Within such short distances, magnetic forces are very strong. A magnet 132 is loaded on a trocar 109, as indicated in FIG. 66. A pair of magnets 132 arranged with polarities attracting each other, as depicted in FIG. 67, is delivered one magnet 132 at a time with a similar delivery device 107 into the urethral 101 wall. To facilitate stress closure by the pulling of the urethropelvic ligament 102 upon the lateral 131 walls, the preferred locations of the magnets 132 are within the posterior 119 and anterior 118 walls, sandwiching the lumen 100, attracting each other to compress and close the lumen 100, as shown in FIG. 68. To void, the anterior 118 muscles stiffen and pull away from the posterior 119 wall, pulling the magnets 132 apart to urinate. After voiding, the muscles relax and the lumen 100 is again compressed and closed by the attraction of two long-lasting magnets 132.

To improve biocompatibility within the urethra 101, the magnets 132 can be coated with polytetrafluoroethylene, silicone, polypropylene, polyethylene, polyurethane, poly-ether-ketone, DELRIN (acetal resin), polysulfone, polycarbonate, polyimide or other coating materials. For visibility, echogenic, radiopaque or other types of coatings can be used.

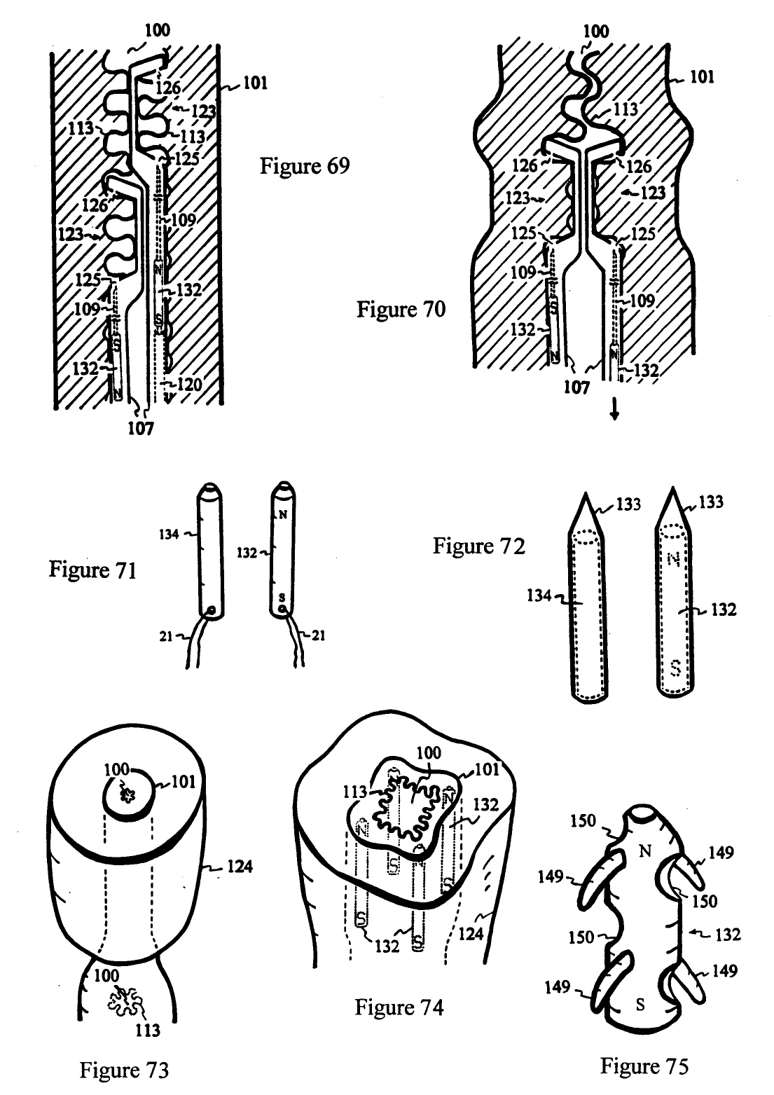

To ensure proper alignment of two attracting magnets 132, two delivery devices 107 loaded with magnets 132 are inserted into the urethra 101, as indicated in FIG. 69, above and below each other with the recesses 123 facing opposite directions. As both recesses 123 align back to back within the urethra 101, as indicated in FIG. 70, the cross section of the aligned recesses 123 greatly increases in diameter, pressing both indented recesses 123 into the soft mucosa 113. This allows both trocars 109 to penetrate and deliver the magnets 132 beneath the surface of mucosa 113, directly across the lumen 100 from each other, as shown in FIG. 70.

Similar to the arch 151, 104 attached to a suture 21, the magnets 132 or the swellable device 134 can also be attached to a suture 21 for device retrieval, as shown in FIG. 71.

Multiple devices 151, 104, 134, 135, 132 utilize the needle 152 or trocar 109 to deliver implants beneath the mucosa 113. It is also possible to implant some of the devices 134, 132 without the needle 152 or trocar 109, by covering the devices 134, 132 with a coating 133, shaped to penetrate the mucosa 113, as shown in FIG. 72. The coating 133 is preferably biodegradable, such as poly-lactate, poly-glycolic or other biodegradable material. After the device 134, 132 has been delivered, the coating 133 degrades and the sharp feature erodes for a safe and effective implant.

### Benefits of the Sphincteric Closure Devices

The device 151, 104, 134, 135 or 132 previously discussed is designed to narrow and/or close the lumen 100 to treat Types 0, I, II and III urinary incontinence, using micro-invasive techniques with the delivery device 107. The device 151, 104, 134, 135 or 132 can also provide the backboard support to the posterior 119 wall to increase urethral resistance, similar to the surgical vaginal 114 repositioning or sling procedure. But unlike invasive surgical procedures, the device 151, 104, 134, 135 or 132 is micro-invasively implanted in the urethral 101 wall, without involving the vagina 114; therefore it is applicable to both men and childbearing women. Furthermore, the range or intensity of lumen 100 closure by the device 151, 104, 134, 135 or 132 is predetermined, measured and limited to avoid urine retention, the most common and unpredictable complication from excessive suture 21 tightening in current surgical procedures.

Similar to the filling location of the bulking agent 140 injection, the preferred device 151, 104, 134 or 135 location is within the posterior wall 119 to allow the muscle-rich anterior 118 wall to widen, control and initiate urination. However, unlike the bulking agents 140, the device 151, 104, 134 or 135 is too large to disperse, too inert to degrade and is accurately delivered to avoid repeating painful injections.

Both the prior art artificial sphincters and the sphinteric closure device 151, 104, 134, 135 or 132 are designed to close the lumen 100 for treating Type III urinary incontinence. However, the insertion of existing artificial sphincters is very invasive, with numerous common complications, such as tube kinking, bladder neck necrosis and infection. Furthermore, the prior art artificial sphincter is manually operated, while the device 151, 104, 134, 135 or 132 in this invention allows the urethral 101 muscles to control urination and assists with lumen 100 closure. Due to the significant size and location of the prior art artificial sphincters, simple activities, such as sitting or bike riding, should be minimized. The micro-artificial sphincteric device 151, 104, 134, 135 or 132 is unlikely to be impacted by daily routines.

Another fine distinction between the implantation of the sphincteric closure device beneath the surface of mucosa 113 and the current surgical corrections is that the device 151, 104, 134, 135 or 132 operates at the sphincteric level, not from outside of the urethral 101 wall. Therefore, the device 151, 104, 134, 135 or 132 is capable of achieving much greater precision, reproducibility and control.

Micro-invasive procedures usually translate into significantly lower costs, shorter recovery times and far fewer complications. Furthermore, the micro-invasive, mini-sphincteric closure device 151, 104, 134, 135 or 132 is suitable for men, older women, and childbearing women, as well as weak patients with minimal to no activity restrictions.

### Opening Urethral Obstruction

The supple texture of smooth muscle 105 and the compliant nature of the urethral 101 wall are crucial elements for successful urethral 101 closure during stress. The compliant urethra 101 is not made to resist external compression or ingrowth of surrounding tissue, such as benign prostatic hyperplasia (BPH). As the prostate 124 grows with time, the urethra 101 is squeezed and the lumen 100 is pinched or even closed, as depicted in FIG. 73.

The polarities of the previously discussed magnets 132 were arranged for attraction, assisting lumen 100 closure in a deficient urethral sphincter. By reversing the magnetic polarities from attracting to repelling, two or more repelling magnets 132 implanted by the delivery device 107 serve to open the pinched lumen 100 by repelling and pushing against the impinging prostatic 124 tissue, as shown in FIG. 74.

In addition to opening the impinged lumen 100 with a micro-invasive procedure, the magnetic devices 132 can be coated or loaded with iodide 125, iodide 131, or other radioactive or chemotherapeutic agent to treat malignant growth in the prostate 124.

Unlike the urethral stents, which are susceptible to clotting by mucosal 113 ingrowth, the magnets 132 are micro-invasively implanted beneath the mucosa 113, unaffected by the fast growing tissue. The close proximity of these magnets provides strong magnetic forces to repel the urethral 101 tissue and to widen the lumen 100. As long as the magnets 132 are properly anchored, the magnetic field will likely provide long-lasting urethral 101 clearance, allowing the urine 117 to flow freely.

To further secure the magnets 132 within the urethral 101 wall, anchoring devices 149 or tissue ingrowth openings 150 can also be added to secure the magnetic 132 device, as depicted in FIG. 75. The anchoring devices 149 can be made with degradable material, allowing time for tissue ingrowth to secure the device 132 before degradation.

### Medical Alert Tags

Most of the devices in this invention are designed to increase urethral resistance by narrowing and/or closing the lumen 100. In hospitals, health care professionals often insert catheters into the urethra 101 for draining. It is possible that the insertion of catheters, especially 12 French or larger, can injure the urethra 101. If the patient has a device 151, 104, 134, 135 or 132, a medical alert tag should be worn.

### Overall Device and Method

Due to the functional similarities of several parts in the delivery devices 107, the names of these parts can be consolidated into generic names. The needle 152 and the trocar 109 can be generally called a trocar or a puncture device. The needle advancer 154 for operating the needle 152 and the trocar advancer 110 for operating the trocar 109 can be generally called a puncture device advancer. The plunger holder 155 and the device advancer 120 can be generally called an implant advancer. The balloon 127 and the recess positioner 142 on the delivery device 107 can be generally called a compressing member. The panel-restricting tube 159 can be generally called a panel deployment delivery device.

The sphincteric closure devices, arch 151, arch tube 104, swellable device 134, inflatable device 135 and magnetic device 132 are all implants. Therefore, the device 151, 104, 134, 135 or 132 can also be called the implant. The main functions of the implants are to treat urinary dysfunctions by altering, reshaping, supporting, restructuring or deforming the urethral tissue.

The methods for delivering the arch 151 with the needle 152 and the arch tube 104 with the trocar 109 are described in detail. By changing the sizes and configurations, the swellable closure device 134, the inflatable device 135 and the magnetic device 132 can also be implanted in the urethral 101 wall with the delivery device 107 using the needle 152 with the plunger 153, or the trocar 109 with the device advancer 120. Multiple devices 151, 104, 134, 135 and/or 132 can also be implanted in series or side by side, using the same or mixed types of devices within a urethra 101.

It is also to be understood that the present invention is by no means limited to the particular constructions disclosed herein and/or shown in the drawings, but also comprises any other modification, changes or equivalents within the scope of the claims. Many features have been listed with particular configurations, options, and embodiments. Any one or more of the features described may be added to or combined with any of the other embodiments or other standard devices to create alternate combinations and embodiments.

It should be clear to one skilled in the art that the current embodiments, materials, constructions, methods, tissues, surgical sites, human or animals, are not the only uses for which the invention may be used. Different materials, shapes, constructions or designs for the arch 104, swellable closure device 134, inflatable closure device 135, magnetic device 132, delivery device 107 and/or recess positioner 142 can be substituted and used. Different methods for delivering the device 151, 104, 134, 135 or 132 can also be modified.

The use of the delivery device 107 is also foreseen for injecting bulking agents 140 accurately into the urethral 101 wall, or narrowing the pylorus or intestine to delay stomach emptying for weight loss purposes. Nothing in the preceding description should be taken to limit the scope of the present invention. The full scope of the invention is to be determined by the appended claims.

We claim:

1. A urethral implant for implantation into a urethral wall for treatment of incontinence, said urethral implant comprising:
an elongated body formed of a resilient material, said body having a first position and a second position, said first position having a curved configuration, said second position being straighten from said first position such that said elongated body is implantable using an implant delivery device,
wherein when said elongated body is placed within a urethral wall, said elongated body is capable of reshaping the urethral wall and thereby facilitating closure of the urethra.

2. The urethral implant of claim 1 wherein said urethral implant has a plurality of curvatures.

3. The urethral implant of claim 1 further comprising a suture connected to an end of said elongated body.

4. The urethral implant of claim 1 wherein said urethral implant has anchoring elements extending from the surface thereof.

5. The urethral implant of claim 1 wherein said urethral implant is formed of a plurality of modular parts.

6. The urethral implant of claim 1 wherein said urethral implant has a circular cross section.

7. The urethral implant of claim 1 wherein said urethral implant has an oval cross section.

8. The urethral implant of claim 1 wherein said urethral implant has an irregular cross section.

9. The urethral implant of claim 1 wherein said urethral implant is formed at least partially of a shape memory material.

10. The urethral implant of claim 1 wherein said urethral implant is formed of a hollow tubular member.

11. The urethral implant of claim 1 wherein said urethral implant is solid.

12. The urethral implant of claim 1 wherein said elongated body is connected to a base member.

13. The urethral implant of claim 12 wherein, in said second position, said base member forms a perimeter around said elongated body.

14. The urethral implant of claim 12 wherein said elongated body is cut out from said base member.

15. The urethral implant of claim 12 wherein, in said first position, said curved configuration of said elongated body protrudes from said base member.

16. The urethral implant of claim 1 wherein said elongated body has a plurality of tissue in-growth holes.

17. The urethral implant of claim 1 wherein said implant is configured such that, when implanted in an operative position within a patient's urethra, said implant is orient such that a longitudinal axis of said implant is aligned with the longitudinal axis of the urethra and a convex side of said implant faces the urethral lumen.

18. The urethral implant of claim 1 further comprising means for orienting said implant with a convex side of said curved configuration oriented toward the urethral lumen.

19. A urethral implant delivery system, comprising:
an implant delivery device, including:
an elongated tubular member having a first end and a second end,
a panel having a front surface and a back surface,
an endwall connecting said panel to said elongated tubular member, said front surface and said endwall forming a recess,
a trocar at least partially extendable into said recess,
a handle connected with said second end of said tubular member,
and a urethral implant sized and configured to fit within said tubular member.

20. The urethral implant delivery system of claim 19 further comprising a deployment opening in said endwall and said trocar extends into said recess through said deployment opening.

21. The urethral implant delivery system of claim 19 further comprising an inflatable balloon located adjacent to said back surface of said panel.

22. The urethral implant delivery system of claim 19 wherein said implant delivery device further comprises a second endwall extending from said panel.

23. The urethral implant delivery system of claim 22 wherein said second endwall has a receiving opening.

24. The urethral implant delivery system of claim 19 wherein said urethral implant has a resilient curve.

25. The urethral implant delivery system of claim 24 wherein said urethral implant is formed of a shape memory material.

26. The urethral implant delivery system of claim 24 wherein said urethral implant is formed of a nickel titanium alloy.

27. The urethral implant delivery system of claim 24 wherein said urethral implant is formed of spring tempered stainless steel.

28. The urethral implant delivery system of claim 24 wherein said urethral implant is less than 5 centimeters in length.

29. The urethral implant delivery system of claim 24 wherein said urethral implant further comprises a leg portion extending from each end of said resilient curve.

30. The urethral implant delivery system of claim 29 wherein said leg portions are generally straight.

31. The urethral implant delivery system of claim 24 wherein said urethral implant is formed from a plurality of modular parts.

32. The urethral implant delivery system of claim 24 wherein said urethral implant is hollow.

33. The urethral implant delivery system of claim 32 wherein said urethral implant has a non-round passage extending therethrough.

34. The urethral implant delivery system of claim 24 further comprising a suture attached to said urethral implant.

35. The urethral implant delivery system of claim 24 wherein said urethral implant is formed from a material chosen from the group consisting of polypropylene, polyethylene, polyurethane, poly-ether-ketone, DELRIN (acetal resin), polysulfone, polycarbonate, polyimide, polytetrafluoroethylene, poly-lactate and poly-glycolic.

36. The urethral implant delivery system of claim 24 wherein said urethral implant has a coating chosen from the group consisting of antibiotic, hormone, growth factor, analgesic, blood clotting, nutrient, radiopaque, echogenic, lubricious, swelling and plasma coatings.

37. The urethral implant delivery system of claim 19 wherein said urethral implant has a plurality of curves.

38. The urethral implant delivery system of claim 19 wherein said urethral implant is swellable.

39. The urethral implant delivery system of claim 38 wherein said urethral implant is formed of a material chosen from the group of materials consisting of collagen, hyaluronate and polyethylene glycol.

40. The urethral implant delivery system of claim 38 wherein said urethral implant has a coating chosen from the group of coatings consisting of antibiotic, hormone, growth factor, analgesic, blood clotting, nutrient, radiopaque, echogenic and lubricious coatings.

41. The urethral implant delivery system of claim 19 wherein said urethral implant has a chamber located therein and is inflatable.

42. The urethral implant delivery system of claim 41 wherein said urethral implant has a one-way valve extending into said chamber for inflation thereof.

43. The urethral implant delivery system of claim 41 wherein said urethral implant is inflatable with a material chosen from the group of materials consisting of silicone oil, air, gas and water.

44. The urethral implant delivery system of claim 41 wherein said urethral implant is formed of an elastic material.

45. The urethral implant delivery system of claim 41 wherein said urethral implant is foldable to allow said implant to fit within a delivery lumen.

46. The urethral implant delivery system of claim 41 wherein said urethral implant is formed from a material chosen from the group of materials consisting of silicon and polyurethane.

47. The urethral implant delivery system of claim 19 wherein said urethral implant has magnetic properties and further comprising a second magnetic implant.

48. The urethral implant delivery system of claim 47 wherein said magnetic urethral implants further comprising a suture.

49. The urethral implant delivery system of claim 47 wherein said magnetic urethral implants further comprising an anchoring device.

50. The urethral implant delivery system of claim 47 wherein said magnetic urethral implants further comprising a tissue ingrowth opening.

51. The urethral implant delivery system of claim 47 wherein said magnetic implants have opposite poles on first ends thereof, whereby the implants attract one another.

52. The urethral implant delivery system of claim 47 wherein said magnetic implants have like poles on first ends thereof, whereby the implants repel one another.

53. The urethral implant delivery system of claim 47 wherein said implant has a coating chosen from the group consisting of polytetrafluoroethylene, silicone, polypropylene, polyethylene, polyurethane, poly-ether-ketone, DELRIN (acetal resin), polysulfone, polycarbonate, polyimide, echogenic, radiopaque material, poly-lactate, poly-glycolic, iodide 125, iodide 131, radioactive material and chemotherapeutic agent.

54. The urethral implant delivery system of claim 19 wherein said trocar has a sharp tip.

55. The urethral implant delivery system of claim 54 wherein said trocar is beveled such that said sharp tip is adjacent said panel.

56. The urethral implant delivery system of claim 19 wherein said trocar has a passage extending therethrough, said passage being sized and configured to contain at least one of said urethral implant.

57. The urethral implant delivery system of claim 56 further comprising an implant advancer located at least partially within said trocar.

58. The urethral implant delivery system of claim 56 wherein said passage is non-round.

59. The urethral implant delivery system of claim 54 wherein said trocar is sized and configured to pass through a passage extending through said urethral implant.

60. The urethral implant delivery system of claim 59 further comprising a urethral implant advancer located around said trocar.

61. The urethral implant delivery system of claim 59 wherein the cross section of said trocar is non-round.

62. The urethral implant delivery system of claim 19 wherein said tubular member has at least one orientation line visible on a surface thereof.

63. The urethral implant delivery system of claim 19 wherein said tubular member has at least one penetration marker visible on a surface thereof.

64. The urethral implant delivery system of claim 19 further comprising a compressing member located adjacent to said back surface of said panel.

65. The urethral implant delivery system of claim 64 wherein said compressing member is sized and configured to be locatable adjacent to said back surface of said panel and to compress the mucosa.

66. The urethral implant delivery system of claim 19 further comprising a second endwall attached to a distal end of said panel, said second endwall having a receiving trough located therein.

67. A urethral implant delivery system, comprising:
a delivery device including:
an elongated tubular member having a first end and a second end,
a panel having a first end portion connecting said panel to said elongated tubular member,
said panel having a first position and a second position, said first position having a curved configuration forming a recess, said second position being straightened from said first position,
and a trocar at least partially extendable into said recess,
and a urethral implant sized and configured to fit within said tubular member.

68. The urethral implant delivery system of claim 67 wherein said panel has a receiving opening in a second end portion thereof.

69. The urethral implant delivery system of claim 68 wherein said receiving opening is a generally round hole extending into said second end portion of said panel.

70. The urethral implant delivery system of claim 68 wherein said receiving opening is a trough extending through said second end portion of said panel.

71. The urethral implant delivery system of claim 67 wherein said panel is formed of a resilient material.

72. The urethral implant delivery system of claim 67 wherein said panel is formed of a shape memory material.

73. The urethral implant delivery system of claim 67 wherein said panel is formed of a material chosen from the group consisting of nickel titanium alloy, spring tempered stainless steel, polypropylene, polyethylene and polyurethane.

74. The urethral implant delivery system of claim 67 further comprising a deployment opening in said first end portion.

75. The urethral implant delivery system of claim 67 further comprising a deployment delivery device located at least partially around said delivery device.

76. The urethral implant delivery system of claim 75 wherein said panel is moved from said second position to said first position by said deployment delivery device.

77. The urethral implant delivery system of claim 67 wherein said panel is moved from said second position to said first position by a change in temperature.

78. A method of micro-invasively treating a dysfunction of the urinary tract, the method comprising the steps of:
(a) inserting an implant delivery device into the urethra of a patient;
(b) orienting a recess of said implant delivery device adjacent to tissue at a first location;
(c) pressing said recess against the tissue, thereby causing a portion of the tissue to enter said recess;
(d) deploying a trocar into said recess;
(e) deploying an implant within a urethral wall located within said recess;
(f) retracting said trocar;
(g) and removing said implant delivery device, leaving said implant within the urethral wall.

79. The method of micro-invasively treating a dysfunction of the urinary tract of claim 78 wherein step (c) is accomplished by inflating a balloon located adjacent to a back surface of said implant delivery device.

80. The method of micro-invasively treating a dysfunction of the urinary tract of claim 78 wherein step (c) is accomplished by positioning a tissue compressor adjacent to a back side of said implant delivery device.

81. The method of micro-invasively treating a dysfunction of the urinary tract of claim 78 wherein step (c) is accomplished by allowing a resilient panel to form a curved configuration to create said recess.

82. The method of micro-invasively treating a dysfunction of the urinary tract of claim 78 wherein step (f) allows said implant to resiliently return to a curved configuration, thereby promoting closure of the urethral passage.

83. The method of micro-invasively treating a dysfunction of the urinary tract of claim 78 wherein said implant is delivered beneath the mucosa.

84. The method of micro-invasively treating a dysfunction of the urinary tract of claim 78 wherein said implant is delivered to a posterior wall of the urethra.

85. The method of micro-invasively treating a dysfunction of the urinary tract of claim 78, further comprising the step of:
(h) allowing said implant to swell, thereby closing the urethral passage.

86. The method of micro-invasively treating a dysfunction of the urinary tract of claim 78, further comprising the step of:
(h) inflating said implant, thereby closing the urethral passage.

87. The method of micro-invasively treating a dysfunction of the urinary tract of claim 78 wherein steps (c) through (f) are repeated at a second location within the urethra.

88. The method of micro-invasively treating a dysfunction of the urinary tract of claim 87 wherein said second location is across the urethral lumen from said first location.

89. The method of micro-invasively treating a dysfunction of the urinary tract of claim 88, further comprising the step of:
(h) allowing a magnetic attraction between said implants at said first and second locations to close the urethral passage.

90. The method of micro-invasively treating a dysfunction of the urinary tract of claim 88, further comprising the step of:
(h) allowing a magnetic repelling force between said implants at said first and second locations to open the urethral passage.

91. The method of micro-invasively treating a dysfunction of the urinary tract of claim 78 wherein steps (b) through (f) are repeated at second, third and fourth locations within the urethra.

92. The method of micro-invasively treating a dysfunction of the urinary tract of claim 91, further comprising the step of:
(h) allowing magnetic repelling force between said implants to open the urethral passage.
